SEQUENCE LISTING (44 sequences)

SEQ ID NO: 1 - protein, 206 aa, Staphylococcus aureus
MKKWTNRLMTIAGVVLILVAAYLFAKPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVK

SEQ ID NO: 2 - protein, 150 aa, Artificial Sequence
  Synthetic Polypeptide
MQAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVKLE

SEQ ID NO: 3 - protein, 149 aa, Artificial Sequence
  Synthetic Polypeptide
QAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVKLE

SEQ ID NO: 4 - protein, 5 aa, Artificial Sequence
  Synthetic Polypeptide
LPETG

SEQ ID NO: 5 - protein, 8 aa, Artificial Sequence
  Synthetic Polypeptide
GGGLPETG

SEQ ID NO: 6 - protein, 5 aa, Artificial Sequence
  Synthetic Polypeptide
GGGGG

SEQ ID NO: 7 - protein, 4 aa, Artificial Sequence
  Synthetic Polypeptide
GGGG

SEQ ID NO: 8 - protein, 6 aa, Artificial Sequence
  Synthetic Polypeptide
GGGGGK

SEQ ID NO: 9 - protein, 156 aa, Staphylococcus aureus
MQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVKLEHHHHHH

SEQ ID NO: 10 - protein, 156 aa, Artificial Sequence
  Synthetic Polypeptide
MQAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVKLEHHHHHH

SEQ ID NO: 11 - protein, 156 aa, Artificial Sequence
  Synthetic Polypeptide
MQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRNVKPTDVEVLDEQKGKDKQLTLITCDDYNEKTGVWEKRKIFVATEVKLEHHHHHH

SEQ ID NO: 12 - protein, 156 aa, Artificial Sequence
  Synthetic Polypeptide
MQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTAVEVLDEQKGKDKQLTLITCDDYNEKTGVWEKRKIFVATEVKLEHHHHHH

SEQ ID NO: 13 - protein, 156 aa, Artificial Sequence
  Synthetic Polypeptide
MQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWETRKIFVATEVKLEHHHHHH

SEQ ID NO: 14 - protein, 156 aa, Artificial Sequence
  Synthetic Polypeptide
MQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRNVKPTDVEVLDEQKGKDKQLTLITCDDYNEETGVWETRKIFVATEVKLEHHHHHH

SEQ ID NO: 15 - protein, 156 aa, Artificial Sequence
  Synthetic Polypeptide
MQAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRDVKPTAVEVLDEQKGKDKQLTLITCDDYNEKTGVWEKRKIFVATEVKLEHHHHHH

SEQ ID NO: 16 - protein, 156 aa, Artificial Sequence
  Synthetic Polypeptide
MQAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRNVKPTAVEVLDEQKGKDKQLTLITCDDYNEKTGVWETRKIFVATEVKLEHHHHHH

SEQ ID NO: 17 - protein, 156 aa, Artificial Sequence
  Synthetic Polypeptide
MQAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRNVKPTDVEVLDEQKGKDKQLTLITCDDYNEKTGVWETRKIFVATEVKLEHHHHHH

SEQ ID NO: 18 - protein, 156 aa, Artificial Sequence
  Synthetic Polypeptide
MQAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRNVKPTAVEVLDEQKGKDKQLTLITCDDYNEKTGVWEKRKIFVATEVKLEHHHHHH

SEQ ID NO: 19 - protein, 156 aa, Artificial Sequence
  Synthetic Polypeptide
MQAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVKLEHHHHHH

SEQ ID NO: 20 - protein, 5 aa, Artificial Sequence
  Synthetic Polypeptide
LAXTG

SEQ ID NO: 21 - protein, 5 aa, Artificial Sequence
  Synthetic Polypeptide
MPXTG

SEQ ID NO: 22 - protein, 5 aa, Artificial Sequence
  Synthetic Polypeptide
LAXSG

SEQ ID NO: 23 - protein, 5 aa, Artificial Sequence
  Synthetic Polypeptide
NPXTG

SEQ ID NO: 24 - protein, 5 aa, Artificial Sequence
  Synthetic Polypeptide
NAXTG

SEQ ID NO: 25 - protein, 5 aa, Artificial Sequence
  Synthetic Polypeptide
NAXSG

SEQ ID NO: 26 - protein, 5 aa, Artificial Sequence
  Synthetic Polypeptide
LPXPG

SEQ ID NO: 27 - protein, 5 aa, Artificial Sequence
  Synthetic Polypeptide
LPXTA

SEQ ID NO: 28 - protein, 5 aa, Artificial Sequence
  Synthetic Polypeptide
LPXTG

SEQ ID NO: 29 - protein, 58 aa, Artificial Sequence
  Synthetic Polypeptide
KKWTNRLMTIAGVVLILVAAYLFAKPHIDNYLHDKDKDEKIEQYDKNVKEQASKDKKQ

SEQ ID NO: 30 - protein, 5 aa, Artificial Sequence
  Synthetic Polypeptide
LPESG

SEQ ID NO: 31 - protein, 5 aa, Artificial Sequence
  Synthetic Polypeptide
LAETG

SEQ ID NO: 32 - protein, 5 aa, Artificial Sequence
  Synthetic Polypeptide
LAESG

SEQ ID NO: 33 - protein, 575 aa, Homo sapiens
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHSGLLIGISIASLCLVVALLALLCHLRKKQGAARAKMEYKCAAPSKEVVLQHVRTERTPQRL

SEQ ID NO: 34 - protein, 577 aa, Mus musculus
MLGIFFLGVLAPASLGLSALAKLQPTGSQCVEHECFALFQGPATFLDASQACQRLQGHLMTVRSSVAADVISLLLSQSSMDLGPWIGLQLPQGCDDPVHLGPLRGFQWVTGDNHTSYSRWARPNDQTAPLCGPLCVTVSTATEAAPGEPAWEEKPCETETQGFLCEFYFTASCRPLTVNTRDPEAAHISSTYNTPFGVSGADFQTLPVGSSAAVEPLGLELVCRAPPGTSEGHWAWEATGAWNCSVENGGCEYLCNRSTNEPRCLCPRDMDLQADGRSCARPVVQSCNELCEHFCVSNAEVPGSYSCMCETGYQLAADGHRCEDVDDCKQGPNPCPQLCVNTKGGFECFCYDGYELVDGECVELLDPCFGSNCEFQCQPVSPTDYRCICAPGFAPKPDEPHKCEMFCNETSCPADCDPNSPTVCECPEGFILDEGSVCTDIDECSQGECFTSECRNFPGSYECICGPDTALAGQISKDCDPIPVREDTKEEEGSGEPPVSPTPGSPTGPPSARPVHSGVLIGISIASLSLVVALLALLCHLRKKQGAARAELEYKCASSAKEVVLQHVRTDRTLQKF

SEQ ID NO: 35 - protein, 6 aa, Artificial Sequence
  Synthetic Polypeptide
LPETGG

SEQ ID NO: 36 - protein, 9 aa, Artificial Sequence
  Synthetic Polypeptide
LPETGGGGG

SEQ ID NO: 37 - protein, 7 aa, Artificial Sequence
  Synthetic Polypeptide
LPETGGG

SEQ ID NO: 38 - protein, 8 aa, Artificial Sequence
  Synthetic Polypeptide
LPETGGGG

SEQ ID NO: 39 - protein, 4 aa, Artificial Sequence
  Synthetic Polypeptide
LPET

SEQ ID NO: 40 - protein, 6 aa, Artificial Sequence
  Synthetic Polypeptide
HHHHHH

SEQ ID NO: 41 - protein, 7 aa, Artificial Sequence
  Synthetic Polypeptide
LAETGGG

SEQ ID NO: 42 - protein, 11 aa, Artificial Sequence
  Synthetic Polypeptide
LAETGHHHHHH

SEQ ID NO: 43 - protein, 7 aa, Artificial Sequence
  Synthetic Polypeptide
LPESGGG

SEQ ID NO: 44 - protein, 7 aa, Artificial Sequence
  Synthetic Polypeptide
LPESGGG

(19) United States
(12) Patent Application Publication
(10) Pub. No.: US 2015/0284477 A1
(43) Pub. Date: Oct. 8, 2015

(54) SORTASE-CATALYZED IMMOBILIZATION, RELEASE, AND REPLACEMENT OF FUNCTIONAL MOLECULES ON SOLID SURFACES

(71) Applicants: President and Fellows of Haverd College, Cambridge, MA (US); Beth Israel Deaconess Medical Center, Inc., Boston, MA (US)

(72) Inventors: Elliot Chaikof, Newton, MA (US); Zheng Qu, Boston, MA (US); Carolyn Haller, Wellesley, MA (US); David R. Liu, Lexington, MA (US); Brent M. Dorr, Somerville, MA (US)

(21) Appl. No.: 14/439,787

(22) PCT Filed: Oct. 30, 2013
(86) PCT No.: PCT/US2013/067461

§ 371 Date: Apr. 30, 2015

Related U.S. Application Data
(60) Provisional application No. 61/720,294, filed on Oct. 30, 2012.

(51) Int. Cl. C07K 17/08; C12N 9/52; C08G 81/00; A61L 33/06; A61L 33/18; C12P 21/00; A61L 33/00

(52) U.S. Cl. CPC C07K 17/08; C12P 21/00; C12N 9/52; A61L 33/0082; A61L 33/068; A61L 33/0041; A61L 33/18; C08G 81/00; C12Y 304/2207; A61L 2300/42; A61L 2400/18

(57) ABSTRACT

Compositions, reagents, kits, and methods for the reversible, covalent conjugation of functional molecules to engineered surfaces, e.g., surfaces of biomedical devices or used in analytical assays or industrial catalysis, are provided. The compositions, reagents, kits, and methods provided herein allow for the attachment, release, and replacement of functional molecules to and from engineered surfaces in vitro, in situ, and in vivo. Implantable vascular grafts, stents, catheters, and other medical devices with immobilized thrombomodulin-coated surfaces are provided. These molecules can be released from the device surface and replaced with fresh thrombomodulin via systemic administration of the respective reactants and without the need to remove the device.

### RELATED APPLICATIONS

This application claims priority under 35 U.S.C. §119(e) to U.S. provisional patent application, U.S. Ser. No. 61/720,294, filed Oct. 30, 2012, the entire contents of which are incorporated herein by reference.

### GOVERNMENT SUPPORT

This invention was made with U.S. Government support under grant 7R01HL056819-13 awarded by the National Institutes of Health. The U.S. Government has certain rights in this invention.

### BACKGROUND OF THE INVENTION

Functional molecules immobilized on a solid support are used in many biotechnological and biomedical fields, including, for example, implantable medical devices (e.g., artificial vascular or tissue grafts), industrial catalysis (e.g., glucose conversion to fructose), analytical devices (e.g., binding resins or columns), and cell and tissue engineering (e.g., cell culture vessels). Immobilized functional molecules often degrade over time, e.g., due to stresses associated with the process or environment they are employed in, e.g., oxidative and enzymatic stresses in vivo, temperature and pH stresses in industrial catalysis, or repeated cycles of binding and release of bound analytes in analytic devices, which limits the useful life span of devices which include such immobilized molecules.

### SUMMARY OF THE INVENTION

A bioinspired surface engineering technology that utilizes reversible sortase-catalyzed transpeptidation to conjugate functional molecules to engineered solid surfaces and to regenerate such immobilized molecules by a repeatable charge/strip cycle has been developed. Some embodiments of the technology disclosed herein utilize the Staphylococcus aureus sortase A (SrtA), which catalyzes covalent transpeptidation of a sortase recognition motif, e.g., LPXT, on substrate proteins to polyglycine nucleophiles. Sortase recognition motif-tagged functional molecules, e.g., enzymes, binding agents, or molecular probes, can be generated by recombinant protein engineering, peptide synthesis techniques, or other methods and then be immobilized on solid supports comprising a compatible sortase recognition motif in vitro, in situ, or in vivo, e.g., on the surface of catheters, stents, vascular grafts, cardiac valves, or other implantable medical devices, on therapeutic or diagnostic particles, on analytical resins or columns, or on cell culture surfaces. One exemplary embodiment is illustrated in FIG. 1.

Some aspects of this disclosure provide a “rechargeable” covalent surface engineering technology that enables regeneration of depleted or degraded biomolecules, e.g., at an interface between a body fluid (e.g., blood) and a medical device in vitro, in situ, or in vivo. This technology has been found to be highly specific and efficient. Due to the synthetic simplicity of conjugating or fusing sortase recognition motifs to functional biomolecules or other molecules such as small molecules, the SrtA-catalyzed transpeptidation technology provided herein can be employed in a range of biotechnology applications including, but not limited to, therapeutic, diagnostic, and analytical applications.

Immobilization of functional molecules, e.g., bioactive agents, on medical device surfaces to control or modulate the host response at device-host interfaces is revolutionizing the design of implantable medical devices, diagnostics, and artificial organ systems. Such surface modifications, e.g., heparin-coating of catheters and vascular grafts, have improved the clinical performance of these devices. However, long term performance of immobilized functional molecules, e.g., enzymes, has been limited due to the degradation of catalytic activity in the operating environment, e.g., by stresses that include, among others, oxidation, hydrolysis, and proteolysis. Some aspects of this disclosure provide a sortase-mediated transpeptidation technology that facilitates regeneration of degraded functional molecules, e.g., enzymes, immobilized on a solid support, such as the surface of a medical device, by a repeatable strip/recharge process.

In some embodiments, the technology provided herein can be employed to facilitate site-specific immobilization of functional molecules conjugated to a sortase recognition motif, e.g., recombinant enzymes fused to a C-terminal LPXT motif to maximize the bioactivity, to strip degraded functional molecules immobilized on an engineered solid support in vitro, in situ, or in vivo, while regenerating sortase recognition motifs on the solid support, and to attach “fresh” functional molecules, e.g., fresh enzymes fused to a C-terminal LPXT motif, to the regenerated sortase recognition motifs on the solid support, thus regenerating the functionality of the solid support.

Due to the synthetic simplicity of incorporating sortase recognition motifs, e.g., oligoglycine and LPXT motifs, on molecules and surfaces, the SrtA-catalyzed transpeptidation technology provided herein is broadly applicable to any field that uses molecules immobilized on a solid support. Such fields include the medical device, therapeutic, analytical, cell and tissue culture, industrial catalysis, and bioprocess engineering fields.

The transpeptidation technology described herein employs a sortase to immobilize functional molecules on a solid surface, and to release and replace such immobilized functional molecules. Wild type (WT) SrtA exhibits low catalytic activity, which necessitates high molar excess of the enzyme as well as longer incubation times, rendering WT SrtA unsuitable for some embodiments, in which lower concentrations of the enzyme or shorter reaction times are desired or required. In some embodiments, an evolved sortase A variant, e.g., a pentamutant SrtA (5′ SrtA) variant, which exhibits a 140-fold higher LPETG (SEQ ID NO: 4)-ligation activity than WT SrtA is employed. The 5′Srt A variant, and additional Srt A variants useful in the context of this disclosure are described in Liu et al., U.S. provisional Patent Application 61/662,606, filed on Jun. 21, 2012, and in Liu et al., U.S. provisional Patent Application 61/880,515, filed on Sep. 20, 2013, the entire contents of each of which are incorporated herein by reference. The use of such enhanced SrtA mutants is typically preferred in embodiments featuring ex vivo or in vivo modification of surfaces.

Some aspects of this disclosure provide a composition comprising an engineered solid support, and a first sortase recognition motif conjugated to the surface of the solid support. In some embodiments, the solid support comprises a polymer or copolymer. In some embodiments, the polymer or co-polymer is chosen from the group consisting of polyurethane polymers and copolymers, polyoxymethylene polymers and copolymers, polyamide polymers and copolymers, polyacrylamide polymers and copolymers, polyvinyl polymers and copolymers, styrene-ethylene-butylene copolymers, styrene-isoprene copolymers, polyether polymers and copolymers, polyolefin polymers and copolymers, polypropylene polymers and copolymers, polyethylene polymers and copolymers, polytetrafluoroethylene (PTFE) polymers and copolymers, polyoxypropylene polymers and copolymers, polyoxyethylene polymers and copolymers, polyanhydride polymers and copolymers, polyvinylalcohol polymers and copolymers, and polyethyleneamine polymers and copolymers. In some embodiments, the first sortase recognition motif comprises a polyglycine sequence. In some embodiments, the polyglycine sequence comprises two or more contiguous glycine residues. In some embodiments, the polyglycine sequence comprises 2, 3, 4, 5, 6, 7, 8, 9, or 10 contiguous glycine residues. In some embodiments, the first sortase recognition motif comprises an LPXT motif, wherein X represents any amino acid. In some embodiments, the first sortase recognition motif comprises an LPETG (SEQ ID NO: 4) motif. In some embodiments, the solid support can be conjugated to a functional molecule conjugated to a second sortase recognition motif via a sortase-mediated transpeptidation, wherein the second sortase recognition motif can be used by a sortase as a substrate of a transpeptidation reaction involving the first sortase recognition motif. In some embodiments, the solid support is conjugated to a functional molecule comprising a second sortase recognition motif. In some embodiments, the composition comprises a [solid support]-[polyglycine sequence]-[LPXT]-[functional molecule] structure, or a [solid support]-[LPXT]-[polyglycine sequence]-[functional molecule] structure. In some embodiments, the solid support is in contact with a body fluid or tissue of a subject. In some embodiments, the solid support is in contact with blood. In some embodiments, the functional molecule has anti-coagulant activity. In some embodiments, the functional molecule is thrombomodulin. In some embodiments, the composition is an implantable medical device. In some embodiments, the composition is a catheter, stent, vascular graft, or cardiac valve. In some embodiments, the medical device comprises a polymeric solid support conjugated to a sortase recognition motif comprising a polyglycine sequence of 2, 3, 4, 5, 6, 7, 8, 9, or 10 contiguous glycine residues. In some embodiments, the medical device comprises a polymeric solid support conjugated to a sortase recognition motif comprising a polyglycine sequence of 2, 3, 4, 5, 6, 7, 8, 9, or 10 contiguous glycine residues, and a functional molecule that inhibits or prevents blood coagulation covalently bound to the polyglycine sequence via an LPXT sortase recognition motif, thus forming a structure: [solid support]-[polyglycine sequence]-[LPXT]-[functional molecule]. In some embodiments, the composition is in contact with a cell or cell population. In some embodiments, the composition is in contact with a cell population in culture. In some embodiments, the composition is comprised in a cell or tissue culture vessel. In some embodiments, the functional molecule is a cell adhesion molecule. In some embodiments, the functional molecule is laminin or fibronectin. In some embodiments, the composition is in contact with an analyte in a sample. In some embodiments, the analyte is a protein, a carbohydrate, a nucleic acid, a lipid, or a small molecule. In some embodiments, the sample is a biological sample. In some embodiments, the sample is obtained from a subject. In some embodiments, the sample is a body fluid or tissue sample. In some embodiments, the sample is a blood, serum, blood plasma, lymph, ascites, urine, saliva, tissue culture, tissue culture supernatant, cell, or tissue sample. In some embodiments, the functional molecule is a binding molecule that specifically binds the analyte. In some embodiments, the functional molecule comprises an antibody or an antibody fragment. In some embodiments, the composition is implantable or biocompatible. Some aspects of this disclosure provide a method comprising contacting a composition as described herein with a sortase under conditions for the sortase to catalyze a transpeptidation reaction.

Some aspects of this disclosure provide a composition comprising an engineered solid support and a functional molecule conjugated to the surface of the solid support via sortase-mediated transpeptidation. In some embodiments, the solid support comprises a polymer. In some embodiments, the solid support comprises a polymer or copolymer. In some embodiments, the polymer or co-polymer is chosen from the group consisting of polyurethane polymers and copolymers, polyoxymethylene polymers and copolymers, polyamide polymers and copolymers, polyacrylamide polymers and copolymers, polyvinyl polymers and copolymers, styrene-ethylene-butylene copolymers, styrene-isoprene copolymers, polyether polymers and copolymers, polyolefin polymers and copolymers, polypropylene polymers and copolymers, polyethylene polymers and copolymers, polytetrafluoroethylene (PTFE) polymers and copolymers, polyoxypropylene polymers and copolymers, polyoxyethylene polymers and copolymers, polyanhydride polymers and copolymers, polyvinylalcohol polymers and copolymers, and polyethyleneamine polymers and copolymers. In some embodiments, the composition comprises a [solid support]-[polyglycine sequence]-[LPXT]-[functional molecule] structure, or a [solid support]-[LPXT]-[polyglycine sequence]-[functional molecule] structure. In some embodiments, the solid support is in contact with a body fluid or tissue of a subject. In some embodiments, the solid support is in contact with blood. In some embodiments, the functional molecule has anti-coagulant activity. In some embodiments, the functional molecule is thrombomodulin. In some embodiments, the composition is an implantable medical device. In some embodiments, the functional molecule has anti-fouling activity. In some embodiments, the functional molecule has specific binding activity. In some embodiments, the functional molecule is an antibody or an antigen-binding antibody fragment, or a cell-adhesion ligand.

Some aspects of this disclosure provide a method for functionalizing a solid support surface, comprising contacting a first sortase recognition motif conjugated to a solid support with a functional molecule conjugated to a second sortase recognition motif in the presence of a sortase and under conditions suitable for the sortase to catalyze a transpeptidation reaction conjugating the functional molecule to the solid support. In some embodiments, the solid support is contacted with the functional molecule in the presence of a sortase in vivo. In some embodiments, the solid support is in contact with blood in a subject, and the contacting is via injection of the functional molecule and the sortase into the blood of the subject. In some embodiments, the solid support is in contact with a tissue in a subject, and the contacting is via administration of the functional molecule and the sortase into the tissue or blood of the subject. In some embodiments, the solid support is an implantable medical device. In some embodiments, the implantable medical device is selected from the group consisting of catheters, stents, cardiac valves, vascular grafts, pumps, heart pacemakers, artificial joints, and cardioverter defibrillators. In some embodiments, the solid support is a catheter or stent. In some embodiments, the functional molecule is a molecule that inhibits coagulation, an anti-fouling agent, or a therapeutic agent. In some embodiments, the functional molecule is thrombomodulin. In some embodiments, the first sortase recognition motif is a polyglycine motif, and the second sortase recognition motif is an LPXT motif. In some embodiments, the first sortase recognition motif is an LPXT motif, and the second sortase recognition motif is a polyglycine motif. In some embodiments, the polyglycine motif comprises two or more contiguous glycine residues. In some embodiments, the polyglycine motif comprises 2, 3, 4, 5, 6, 7, 8, 9, or 10 contiguous glycine residues. In some embodiments, the LPXT motif comprises an LPXTG motif, wherein X represents any amino acid. In some embodiments, the LPXT motif comprises an LPETG motif. In some embodiments, the sortase is a sortase comprising an amino acid sequence that is at least 90% homologous to the amino acid sequence of S. aureus Sortase A provided as SEQ ID NO: 1 or a fragment thereof, wherein the amino acid sequence of the sortase comprises one or more mutations selected from the group consisting of P94S, P94R, E106G, F122Y, F154R, D160N, D165A, G174S, K190E, and K196T. In some embodiments, the sortase comprises an amino acid sequence that is at least 95%, at least 98%, or at least 99% homologous to SEQ ID NO: 1 or a fragment thereof. In some embodiments, the amino acid sequence of the sortase comprises at least one mutation, at least two mutations, at least three mutations, or at least four mutations as compared to the amino acid sequence of S. aureus Sortase A provided as SEQ ID NO: 1 or a fragment thereof. In some embodiments, the sortase comprises a P94S or P94R mutation, a D160N mutation, a D165A mutation, a K190E mutation, and a K196T mutation. In some embodiments, the sortase exhibits a k_{cat} that is at least 1.5-fold, at least 2-fold, or at least 3-fold greater than the kcat of the corresponding wild type S. aureus Sortase A amino acid sequence. In some embodiments, the sortase exhibits a K_{M} for a substrate comprising the amino acid sequence LPETG (SEQ ID NO: 4) that is at least 2-fold, at least 5-fold, or at least 10-fold less than the K_{M} of the corresponding wild type sortase A amino acid sequence. In some embodiments, the sortase exhibits a K_{M} for a substrate comprising the amino acid sequence GGG that is not more than 2-fold, not more than 5-fold, not more than 10-fold, or not more than 20-fold greater than the K_{M} of the corresponding wild type sortase A amino acid sequence. In some embodiments, the sortase exhibits a ratio of K_{cal}/K_{M} for a substrate comprising the amino acid sequence LPETG (SEQ ID NO: 4) that is least 2-fold, at least 5-fold, at least 10-fold, at least 20-fold, at least 50-fold, at least 100-fold, or at least 120-fold greater than the K_{cal}/K_{M} ratio of the corresponding wild type sortase A amino acid sequence. In some embodiments, the sortase comprises the sequence:

(SEQ ID NO: 3)
QAKPQIPKDKSKVAGYIEIPDADIKEPVYPGPATREQLNRGVSFAEENES
LDDQNISIAGHTFIDRPNYQFTNLKAAKKGSMVYFKVGNETRKYKMTSIR
NVKPTAVEVLDEQKGKDKQLTLITCDDYNEETGVWETRKIFVATEVKLE,

or a sequence that is at least 90%, at least 95%, at least 98%, or at least 99% identical to the sequence provided in SEQ ID NO: 3.

Some aspects of this disclosure provide a method comprising contacting a first functional molecule conjugated to a solid support comprising a first and a second sortase recognition motif with a sortase in the presence of a peptide comprising the first sortase recognition motif under conditions suitable for the sortase to catalyze a transpeptidation reaction that releases the first functional molecule and the second sortase recognition motif from the solid support and that restores the first sortase recognition motif conjugated to the solid support. In some embodiments, the solid support is contacted with the sortase in the presence of the peptide in vivo. In some embodiments, the solid support is in contact with blood in a subject, and the contacting is via injection of the peptide and the sortase into the blood of the subject. In some embodiments, the solid support is in contact with a tissue in a subject, and the contacting is via administration of the peptide and the sortase into the tissue or blood of the subject. In some embodiments, the solid support is an implantable medical device. In some embodiments, the implantable medical device is selected from the group consisting of catheters, stents, cardiac valves, vascular grafts, pumps, heart pacemakers, artificial joints, and cardioverter defibrillators. In some embodiments, the solid support is a catheter or stent. In some embodiments, the functional molecule is a molecule that inhibits coagulation, an anti-fouling agent, or a therapeutic agent. In some embodiments, the functional molecule is thrombomodulin. In some embodiments, the first sortase recognition motif comprises a polyglycine sequence. In some embodiments, the functional molecule is conjugated to the solid support via a [solid support]-[polyglycine]-[LPXT]-[functional molecule] structure, and wherein the peptide comprises a polyglycine sequence, wherein X represents any amino acid. In some embodiments, the first sortase recognition motif comprises an LPXT sequence wherein X represents any amino acid. In some embodiments, the functional molecule is conjugated to the solid support via a [solid support]-[LPXT]-[polyglycine]-[functional molecule] structure, and wherein the peptide comprises an LPXT sequence wherein X represents any amino acid. In some embodiments, the polyglycine motif comprises two or more contiguous glycine residues. In some embodiments, the polyglycine motif comprises 2, 3, 4, 5, 6, 7, 8, 9, or 10 contiguous glycine residues. In some embodiments, the LPXT motif comprises an LPXTG motif wherein X represents any amino acid. In some embodiments, the LPXT motif comprises an LPETG (SEQ ID NO: 4) motif. In some embodiments, the sortase is a sortase comprising an amino acid sequence that is at least 90% homologous to the amino acid sequence of S. aureus Sortase A provided as SEQ ID NO: 1 or a fragment thereof, wherein the amino acid sequence of the sortase comprises one or more mutations selected from the group consisting of P94S, P94R, E106G, F122Y, F154R, D160N, D165A, G174S, K190E, and K196T. In some embodiments, the sortase comprises an amino acid sequence that is at least 95%, at least 98%, or at least 99% homologous to SEQ ID NO: 1 or a fragment thereof. In some embodiments, the amino acid sequence of the sortase comprises at least one mutation, at least two mutations, at least three mutations, or at least four mutations as compared to the amino acid sequence of S. aureus Sortase A provided as SEQ ID NO: 1 or a fragment thereof. In some embodiments, the sortase comprises a P94S or P94R mutation, a D160N mutation, a D165A mutation, a K190E mutation, and a K196T mutation. In some embodiments, the sortase exhibits a k_{cat} that is at least 1.5-fold, at least 2-fold, or at least 3-fold greater than the kcat of the corresponding wild type S. aureus Sortase A amino acid sequence. In some embodiments, the sortase exhibits a K_{M} for a substrate comprising the amino acid sequence LPETG (SEQ ID NO: 4) that is at least 2-fold, at least 5-fold, or at least 10-fold less than the K_{M} of the corresponding wild type sortase A amino acid sequence. In some embodiments, the sortase exhibits a K_{M} for a substrate comprising the amino acid sequence GGG that is not more than 2-fold, not more than 5-fold, not more than 10-fold, or not more than 20-fold greater than the K_{M} of the corresponding wild type sortase A amino acid sequence. In some embodiments, the sortase exhibits a ratio of K_{cat}/K_{M} for a substrate comprising the amino acid sequence LPETG (SEQ ID NO: 4) that is least 2-fold, at least 5-fold, at least 10-fold, at least 20-fold, at least 50-fold, at least 100-fold, or at least 120-fold greater than the K_{cat}/K_{M} ratio of the corresponding wild type sortase A amino acid sequence. In some embodiments, the sortase comprises the sequence:

(SEQ ID NO: 3)
QAKPQIPKDKSKVAGYIEIPDADIKEPVYPGPATREQLNRGVSFAEENES
LDDQNISIAGHTFIDRPNYQFTNLKAAKKGSMVYFKVGNETRKYKMTSIR
NVKPTAVEVLDEQKGKDKQLTLITCDDYNEETGVWETRKIFVATEVKLE,

or a sequence that is at least 90%, at least 95%, at least 98%, or at least 99% identical to the sequence provided in SEQ ID NO: 3. In some embodiments, the method further comprises contacting the solid support comprising a restored first sortase recognition motif with a sortase and a second functional molecule conjugated to the second sortase recognition motif under conditions suitable for the sortase to catalyze a transpeptidation reaction forming a covalent bond between the first and the second sortase recognition motifs, thus conjugating the second functional molecule to the solid support. In some embodiments, the contacting is in vivo. In some embodiments, the method comprises multiple cycles of releasing a functional molecule from the solid support via sortase-mediated transpeptidation, and conjugating a functional molecule to the solid support via sortase-mediated transpeptidation. In some embodiments, the method comprises at least 2, at least 3, at least 4, at least 5, at least 6, at least 7, at least 8, at least 9, or at least 10 cycles of releasing a functional molecule from the solid support via sortase-mediated transpeptidation, and conjugating a functional molecule to the solid support via sortase-mediated transpeptidation. In some embodiments, the first functional molecule is of the same type as the second functional molecule.

Some aspects of this disclosure provide a kit comprising a functional molecule conjugated to a sortase recognition motif, and a sortase. In some embodiments, the sortase recognition motif conjugated to the functional molecule comprises an LPXT motif, wherein X represents any amino acid. In some embodiments, the kit further comprises a peptide comprising a sortase recognition motif. In some embodiments, the sortase recognition motif comprised in the peptide is a polyglycine motif. In some embodiments, the polyglycine motif comprises 2, 3, 4, 5, 6, 7, 8, 9, or 10 contiguous glycine residues. In some embodiments, the functional molecule, the sortase, and/or the peptide are provided in pharmaceutically acceptable form. In some embodiments, the kit further comprises an injectable solvent.

Some aspects of this disclosure provide a culture vessel comprising a compartment for holding a culture medium and cells, wherein the surface of the compartment facing the culture medium or the cells comprises a sortase recognition motif. In some embodiments, the culture vessel further comprises a functional molecule conjugated to the surface of the compartment facing the culture medium via sortase-mediated transpeptidation. In some embodiments, the functional molecule is a cell adhesion ligand. In some embodiments, the cell adhesion ligand is laminin or fibronectin.

Some aspects of this disclosure provide a device for the isolation of an analyte, comprising a solid support that can be contacted with a sample comprising an analyte, and a sortase recognition motif conjugated to the surface of the solid support. In some embodiments, the column further comprises a functional molecule conjugated to the surface of the solid support via sortase-mediated transpeptidation. In some embodiments, the functional molecule is a binding agent. In some embodiments, the functional molecule is an antibody or an antigen-binding fragment thereof. In some embodiments, the analyte comprises a second sortase recognition motif, and wherein the second sortase recognition motif can participate in a sortase-mediated transpeptidation reaction with the first sortase recognition motif, resulting in covalent binding of the analyte to the column.

The Summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1. Two-step “rechargeable” surface assembly cycle initiated by sortase-catalyzed charging of LPETG (SEQ ID NO: 4) tagged biomolecules on pentaglycine-modified surfaces, followed by stripping to regenerate pentaglycine anchor sites for additional reaction cycles.

FIG. 2. Sortase-catalyzed rechargeable assembly of LPETG (SEQ ID NO: 4)-labeled thrombomodulin (TM_{LPETG}) on pentaglycine-modified model surfaces. (A) Immobilization of 1 μM TM_{LPETG} on pentaglycine-coated microwells using 0.1 molar equivalents evolved 5′ sortase, 0.1 and 2 molar equivalents wild-type (WT) sortase, or no sortase as a negative control. (B) Following immobilization of 1 μM TM_{LPETG} on pentaglycine coated microwells using 0.1 molar equivalents evolved 5′ sortase, removal of bound TM was carried out using 20 μM of either evolved 5′ sortase or WT sortase with 1 mM triglycine.

FIG. 3. Sortase-catalyzed binding of TM_{LPETG} on pentaglycine-modified model surfaces following 1 and 16 hour reactions were compared with the binding of TM-biotin directly on streptavidin-coated microwells. In parallel, TM_{LPETG} was incubated in microwells without sortase as a negative control.

FIG. 4. Sequential 5′ sortase-catalyzed charging (diamonds) and stripping (circles) cycles of TM_{LPETG} performed on model pentaglycine surfaces.

FIG. 5. Direct sortase-catalyzed assembly of TM_{LPETG} in 50% v/v heparinized whole blood (20α heparin/mL blood) at 37° C. for 1 hour without additional calcium. Evolved and wild type (WT) sortases were tested at two different TM_{LPETG} concentrations as well as TM_{LPETG}/sortase ratios, as summarized in the table of reaction conditions.

FIG. 6. (A) Merged fluorescent and bright field microscopy of polyurethane catheters modified with pentaglycine motifs, and reacted with various concentrations of biotin-LPETG (SEQ ID NO: 4) peptide for 30 minutes or 1 hour. Pentamutant sortase was kept at a constant 0.1 molar equivalent ratio relative to biotin-LPETG (SEQ ID NO: 4). Cy3-streptavidin was incubated at 0.1 mg/ml with catheters for 30 minutes to assess the surface density of biotin. (B) Fluorescence intensity was measured using Image J and expressed as mean±std. dev. for three individual catheter segments per reaction condition.

FIG. 7. (A) Merged fluorescent and bright field microscopy of polyurethane catheters modified with pentaglycine motifs, reacted with biotin-LPETG (SEQ ID NO: 4), and finally with various concentrations of GGG peptide and 5′ SrtA as summarized in (C). Cy3-streptavidin was incubated at 0.1 mg/ml with catheters for 30 minutes to assess the surface density of biotin. (B) Fluorescence intensity was measured using Image J and expressed as mean±std. dev. for three individual catheter segments per reaction condition.

FIG. 8. (A) Merged fluorescent and bright field microscopy of polyurethane catheters modified with pentaglycine motifs, reacted with biotin-LPETG (SEQ ID NO: 4), and finally with various concentrations of GGG peptide and 5′ SrtA as summarized in (C). Cy3-streptavidin was incubated at 0.1 mg/ml with catheters for 30 minutes to assess the surface density of biotin. (B) Fluorescence intensity was measured using Image J and expressed as mean±std. dev. for three individual catheter segments per reaction condition.

FIG. 9. Polyurethane catheters are chemically modified with GGG anchor sites for sortase-catalyzed transpeptidation of a biotin-LPETG (SEQ ID NO: 4) peptide probe (reaction 1). These biotinylated catheters were deployed in the aorta of mice and “stripped” (reaction 2) using sortase and GGG peptide. In order to detect the depletion of biotin from the catheter surface, streptavidin-Cy3 was incubated with the tested catheters and detected using fluorescent microscopy. A mouse model was employed to validate reversible modification of the blood-contacting interface of catheters deployed in the mouse vena cava. (A) Illustration of the deployed catheter in the vena cava undergoing charging reaction 1 with sortase and LPETG (SEQ ID NO: 4) tagged probe, and stripping reaction 2 with sortase and triglycine peptide. (B) Representative surgical setup, with anesthetized mouse undergoing cannulation of catheter in the vena cava. (C) A fully deployed catheter in the vena cava with the insertion site highlighted.

FIG. 10. Fluorescent imaging of cannulated catheters subjected to in vivo modification by sortase-catalyzed transpeptidation. Biotin-LPETG (SEQ ID NO: 4) probes were used to functionalize GGG-modified catheters via transpeptidation, and streptavidin-Cy3 was used to detect the presence of biotin. (A) A fully biotinylated catheter, (B) a biotinylated catheter deployed in a mouse with intravenous injection of 400 μg triglycine and 700 μg 5′ SrtA, (C) a GGG-modified catheter deployed in a mouse with intravenous injection of 50 μg biotin-LPETG (SEQ ID NO: 4) and 70 μg 5′ SrtA, (D) a GGG-modified catheter deployed in a mouse with intravenous injection of 250 μg biotin-LPETG (SEQ ID NO: 4) and 350 μg 5′ SrtA. Arrows bracket the insertion length of the catheter into the mouse vena cava.

FIG. 11. Real-time sortase-catalyzed stripping of catheters cannulated in mouse vena cava.

FIG. 12. Baboon shunt model to evaluate the acute blood contacting properties of materials. (A) Exteriorized arteriovenous shunt model in non-human primates (B) thrombosis in a 2-chamber shunt configuration after 1 hour perfusion in the shunt (C) shunt configuration showing the use of a Dacron segment as a thrombotic stimulus upstream of test materials (D) platelet deposition in real time on the Dacron region and (E) on either plain ePTFE control grafts from Gore, Propaten heparin coated grafts from Gore, and TM-modified grafts.

FIG. 13. Sequential modification of a medical device to generate LPETG (SEQ ID NO: 4) motifs, which form a stable acyl-intermediate with sortase modified with anti-fouling components. This coating can be stripped with GGG peptide and recharged with fresh film.

FIG. 14. (A) Affinity chromatography in three steps using sortase-modified solid support; (B) sortase-catalyzed acyl intermediate formation will specifically bind LPETG (SEQ ID NO: 4)-tagged proteins from a crude mixture, and facilitate elution of the desired product using GGG peptide.

FIG. 15. (A) Generation of cell-adhesive surfaces using LPETG (SEQ ID NO: 4)-tagged adhesion ligands or synthetic polymers; (B) Following cell culture, intact cell sheets can be removed by cleaving the sortase linked adhesion protein anchors.

FIG. 16. Activity of evolved sortases.

FIG. 17. Analysis of the sortase mechanism reveals a fourth kinetic parameter.

FIG. 18. Characterization of evolved sortases. Upper panel: 2A sortases. Lower panel: 4S sortases.

FIG. 19. Structure of evolved sortases.

FIG. 20. Tissue slide labeling. The pentamutant sortase (Smut), and two variants (4S.6a and 2A.6a) were compared. Combining a pre-block and a lipofuscin stain significantly improved signal to noise in tissue slide labeling.

FIG. 21. Tissue slide labeling of small intestine.

FIG. 22. Tissue slide labeling of testis.

FIG. 23. Sortase-mediated protein modification overview.

FIG. 24. Sortase-mediated protein modification results. Anti-His6 staining (upper panel) and anti-BTN staining (lower panel).

### DEFINITIONS

The term “agent,” as used herein, refers to any molecule, entity, or moiety. For example, an agent may be a protein, an amino acid, a peptide, a polynucleotide, a carbohydrate, a lipid, a detectable label, a binding agent, a tag, a metal atom, a contrast agent, a catalyst, a non-polypeptide polymer, a synthetic polymer, a recognition element, a linker, or a chemical compound, such as a small molecule. In some embodiments, the agent is a binding agent, for example, a ligand, a ligand-binding molecule, an antibody, or an antibody fragment. Additional agents suitable for use in embodiments of the present invention will be apparent to the skilled artisan. The invention is not limited in this respect.

The term “amino acid,” as used herein, includes any naturally occurring and non-naturally occurring amino acid. Suitable natural and non-natural amino acids will be apparent to the skilled artisan, and include, but are not limited to, those described in S. Hunt, The Non-Protein Amino Acids: In Chemistry and Biochemistry of the Amino Acids, edited by G. C. Barrett, Chapman and Hall, 1985. Some non-limiting examples of non-natural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-l-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; —CH₃), disubstituted phenylalanines, substituted tyrosines (e.g., further substituted with —C(═O)C₆H₅; —CF₃; —CN; -halo; —NO₂; —CH₃), and statine. In the context of amino acid sequences, “X” or “Xaa” represents any amino acid residue, e.g., any naturally occurring and/or any non-naturally occurring amino acid residue.

The term “antibody,” as used herein, refers to a protein belonging to the immunoglobulin superfamily. The terms antibody and immunoglobulin are used interchangeably. Antibodies from any mammalian species (e.g., human, mouse, rat, goat, pig, horse, cattle, camel) and from non-mammalian species (e.g., from non-mammalian vertebrates, birds, reptiles, amphibia) are within the scope of the term. Suitable antibodies and antibody fragments for use in the context of some embodiments of the present invention include, for example, human antibodies, humanized antibodies, domain antibodies, F(ab′), F(ab′)2, Fab, Fv, Fc, and Fd fragments, antibodies in which the Fc and/or FR and/or CDR1 and/or CDR2 and/or light chain CDR3 regions have been replaced by homologous human or non-human sequences; antibodies in which the FR and/or CDR1 and/or CDR2 and/or light chain CDR3 regions have been replaced by homologous human or non-human sequences; antibodies in which the FR and/or CDR1 and/or CDR2 and/or light chain CDR3 regions have been replaced by homologous human or non-human sequences; and antibodies in which the FR and/or CDR1 and/or CDR2 regions have been replaced by homologous human or non-human sequences. In some embodiments, so-called single chain antibodies (e.g., ScFv), (single) domain antibodies, and other intracellular antibodies may be used in the context of the present invention. Domain antibodies, camelid and camelized antibodies, and fragments thereof, for example, VHH domains, or nanobodies, are also encompassed by the term antibody. Further, chimeric antibodies, e.g., antibodies comprising two antigen-binding domains that bind to different antigens, are also suitable for use in the context of some embodiments of the present invention.

The term “binding agent,” as used herein refers to any molecule that binds another molecule. In some embodiments, a binding agent binds another molecule with high affinity. In some embodiments, a binding agent binds another molecule with high specificity. Examples for binding agents include, without limitation, antibodies, antibody fragments, receptors, ligands, aptamers, and adnectins.

The term body fluid, as used herein, refers to any body fluid including, without limitation, blood, serum, plasma, lymph fluid, synovial fluid, follicular fluid, seminal fluid, amniotic fluid, milk, sweat, urine, cerebrospinal fluid, saliva, semen, sputum, tears, perspiration, mucus, and interstitial fluid. The term also applies to fractions and dilutions of body fluids.

The term “conjugated” or “conjugation” refers to an association of two entities, for example, of two molecules such as two proteins, or a protein and a reactive handle, or a protein and an agent, e.g., a detectable label. The association can be, for example, via a direct or indirect (e.g., via a linker) covalent linkage or via non-covalent interactions. In some embodiments, the association is covalent. In some embodiments, two molecules are conjugated via a linker connecting both molecules. For example, in some embodiments where two proteins are conjugated to each other to form a protein fusion, the two proteins may be conjugated via a polypeptide linker, e.g., an amino acid sequence connecting the C-terminus of one protein to the N-terminus of the other protein. In some embodiments, conjugation of a protein to a protein or peptide is achieved by transpeptidation using a sortase. See, e.g., Ploegh et al., International PCT Patent Application, PCT/US2010/000274, filed Feb. 1, 2010, published as WO/2010/087994 on Aug. 5, 2010; Ploegh et al., International Patent Application PCT/US2011/033303, filed Apr. 20, 2011, published as WO/2011/133704 on Oct. 27, 2011; Liu et al., U.S. provisional Patent Application 61/662,606, filed on Jun. 21, 2012; and Liu et al., U.S. provisional Patent Application 61/880,515, filed on Sep. 20, 2013; the entire contents of each of which are incorporated herein by reference, for exemplary sortases, proteins, recognition motifs, reagents, and methods for sortase-mediated transpeptidation.

The term “functional molecule” refers to an agent, e.g., a molecule (e.g., a protein, carbohydrate, nucleic acid, lipid, or small molecule), or moiety, that exhibits a function, e.g., a catalytic activity, a binding activity, a therapeutic activity, or an activity as a detectable label. In some embodiments, a functional molecule is a protein having a catalytic activity, e.g., an enzyme or a fragment thereof. In some embodiments, a functional molecule is a protein having a binding activity, e.g., a binding agent such as an antibody or an antigen-binding antibody fragment. In some embodiments, a functional molecule is a small molecule or a nucleic acid having a binding activity. In some embodiments, a functional molecule is a small molecule, a peptide or protein, or a nucleic acid having a therapeutic activity, e.g., a cytotoxic, cytostatic, anti-angiogenic, or pro-apoptotic activity; or a pro-survival, anti-apoptotic, or pro-angiogenic activity.

The term “homologous”, as used herein is an art-understood term that refers to nucleic acids or polypeptides that are highly related at the level of nucleotide or amino acid sequence. Nucleic acids or polypeptides that are homologous to each other are termed “homologues.” Homology between two sequences can be determined by sequence alignment methods known to those of skill in the art. In accordance with the invention, two sequences are considered to be homologous if they are at least about 50-60% identical, e.g., share identical residues (e.g., amino acid residues) in at least about 50-60% of all residues comprised in one or the other sequence, at least about 70% identical, at least about 80% identical, at least about 90% identical, at least about 95% identical, at least about 98% identical, at least about 99% identical, at least about 99.5% identical, or at least about 99.9% identical, for at least one stretch of at least 20, at least 30, at least 40, at least 50, at least 60, at least 70, at least 80, at least 90, at least 100, at least 120, at least 150, or at least 200 amino acids.

The term “k_{cat}” refers to the turnover rate of an enzyme, e.g., the number of substrate molecules that the respective enzyme converts to product per time unit. Typically, k_{cat} designates the turnover of an enzyme working at maximum efficiency.

The term “K_{M}” is used herein interchangeably with the term “Kₘ” and refers to the Michaelis constant of an enzyme, an art-recognized measure designating the substrate concentration at ½ the maximum reaction velocity of a reaction catalyzed by the respective enzyme.

The term “linker,” as used herein, refers to a chemical group or molecule covalently linked to a molecule, for example, a protein, and a chemical group or moiety. In some embodiments, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer (e.g., PEG), or chemical moiety.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. For example, the term “P94S” in the context of describing a mutation in the S. aureus sortase A protein describes a mutation in which the P (proline) residue at position 94 in the sortase A sequence has been replaced by an S (serine) residue, the term “P94R” describes a mutation in which the P (proline) residue at position 94 in the sortase A sequence has been replaced by an R (arginine) residue, the term “E106G” describes a mutation in which the E (glutamate) residue at position 106 in the sortase A sequence has been replaced by a G (glycine) residue, and so forth. See, e.g., SEQ ID NO: 1 for reference of the respective amino acid residue positions in the wild type S. aureus sortase A protein.

The term “polyglycine,” used herein interchangeably with the term “oligoglycine,” refers to an amino acid sequence comprising or consisting of a plurality of contiguous glycine residues. In some embodiments, a polyglycine peptide comprises or consists of 2, 3, 4, 5, 6, 7, 8, 9, 10, or more than 10 contiguous glycine residues. In some embodiments, a polyglycine peptide comprises or consists of at least 2, at least 3, at least 4, at least 5, at least 6, at least 7, at least 8, at least 9, or at least 10 contiguous glycine residues.

The term “physiological conditions,” as used herein, refers to a range of chemical (e.g., pH, ionic strength), biochemical (e.g., enzyme concentrations), and physical (e.g., temperature, pressure) conditions that can be encountered in intracellular and extracellular fluids of tissues, such as, for example, in the intracellular and extracellular fluids of a subject. For most cells and tissues, the physiological pH ranges from about 7.0 to about 7.5, the physiological ionic strength ranges from about 50 mM to about 400 mM, the physiological temperature ranges from about 20° C. to about 42° C., and the physiological pressure ranges from about 925 mbar to about 1050 mbar. Physiological conditions may include, for example, an aqueous environment, e.g., an exposure to a cell, tissue, or body fluid, or an exposure to air.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

The term “small molecule” is used herein to refer to molecules, whether naturally-occurring or artificially created (e.g., via chemical synthesis) that have a relatively low molecular weight. Typically, a small molecule is an organic compound (i.e., it contains carbon). A small molecule may contain multiple carbon-carbon bonds, stereocenters, and other functional groups (e.g., amines, hydroxyl, carbonyls, or heterocyclic rings). In some embodiments, small molecules are monomeric and have a molecular weight of less than about 1500 g/mol. In certain embodiments, the molecular weight of the small molecule is less than about 1000 g/mol or less than about 500 g/mol. In certain embodiments, the small molecule is a drug, for example, a drug that has already been deemed safe and effective for use in humans or animals by the appropriate governmental agency or regulatory body.

The term “solid support,” as used herein, refers to a water-insoluble substance which is in a solid state under physiological conditions. A solid support may comprise a single type of material, e.g., a single polymeric material, or a plurality of different materials, e.g., a copolymer. Non-limiting solid support materials include various plastics, glasses, and clays, for example, organic polymers and copolymers, SiO₂, and Al₂O₃. In the context of implantable medical devices, a solid support typically comprises a biocompatible material, e.g., a biocompatible polymer, copolymer, or glass, and is free of toxic additives. Suitable polymers and copolymers include, without limitation, e.g., polycarboxylic acid polymers and copolymers, including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyl resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, polystyrenes, styrene-maleic anhydride copolymers, styrene-butadiene copolymers, styrene-ethylene-butylene copolymers (e.g., a polystyrene-polyethylene/butylene-polystyrene (SEBS) copolymer), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); glycosaminoglycans; polyesters including polyethylene terephthalates and aliphatic polyesters such as polymers and copolymers of lactide (which includes lactic acid as well as d-,1- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropr-opene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), polyoxypropylenes, PLURONIC™ family of block copolymers including PLURONIC® F68, PLURONIC® F108, PLURONIC® F127, PLURONIC® F38, PLURONIC® F68, PLURONIC® F77, PLURONIC® F87, PLURONIC® F88, PLURONIC® F98, PLURONIC® L10, PLURONIC® L101, PLURONIC® L121, PLURONIC® L31, PLURONIC® L35, PLURONIC® L43, PLURONIC® L44, PLURONIC® L61, PLURONIC® L62, PLURONIC® L64, PLURONIC® L81, PLURONIC® L92, PLURONIC® N3, PLURONIC® P103, PLURONIC® P104, PLURONIC® P105, PLURONIC® P123, PLURONIC® P65, PLURONIC® P84, and PLURONIC® P85, polyoxyethylene (polyethylene glycol), polyanhydrides, polyvinylalcohol, polyethyleneamine and polypyrridine, as well as blends and copolymers of the above. The polymers and copolymers may be provided in a variety of configurations, including cyclic, linear and branched configurations. Branched configurations include star-shaped configurations (e.g., configurations in which three or more chains emanate from a single branch point), comb configurations (e.g., graft polymers having a main chain and a plurality of branching side chains), and dendritic configurations (e.g., arborescent and hyperbranched polymers). The polymers can be formed from a single monomer (i.e., they can be homopolymers), or they can be formed from multiple monomers (i.e., they can be copolymers) that can be distributed, for example, randomly, in an orderly fashion (e.g., in an alternating fashion), or in blocks. Additional materials suitable as solid supports in the context of this disclosure will be apparent to those of skill in the art. The disclosure is not limited in this respect.

The term “sortase,” as used herein, refers to a protein having sortase activity, i.e., an enzyme able to carry out a transpeptidation reaction conjugating the C-terminus of a protein to the N-terminus of a protein via transamidation. The term includes full-length sortase proteins, e.g., full-length naturally occurring sortase proteins, fragments of such sortase proteins that have sortase activity, modified (e.g., mutated) variants or derivatives of such sortase proteins or fragments thereof, as well as proteins that are not derived from a naturally occurring sortase protein, but exhibit sortase activity. Those of skill in the art will readily be able to determine whether or not a given protein or protein fragment exhibits sortase activity, e.g., by contacting the protein or protein fragment in question with a suitable sortase substrate under conditions allowing transpeptidation and determining whether the respective transpeptidation reaction product is formed. In some embodiments, a sortase is a protein comprising at least 20 amino acid residues, at least 30 amino acid residues, at least 40 amino acid residues, at least 50 amino acid residues, at least 60 amino acid residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues. In some embodiments, a sortase is a protein comprising less than 100 amino acid residues, less than 125 amino acid residues, less than 150 amino acid residues, less than 175 amino acid residues, less than 200 amino acid residues, or less than 250 amino acid residues.

Suitable sortases will be apparent to those of skill in the art and include, but are not limited to, sortase A, sortase B, sortase C, and sortase D type sortases. Suitable sortases are described, for example, in Dramsi S, Trieu-Cuot P, Bierne H, Sorting sortases: a nomenclature proposal for the various sortases of Gram-positive bacteria. Res Microbiol. 156(3):289-97, 2005; Comfort D, Clubb RT. A comparative genome analysis identifies distinct sorting pathways in gram-positive bacteria. Infect Immun., 72(5):2710-22, 2004; Chen I, Dorr B M, and Liu D R., A general strategy for the evolution of bond-forming enzymes using yeast display. Proc Natl Acad Sci USA. 2011 Jul. 12; 108(28):11399; and Pallen, M. J.; Lam, A. C.; Antonio, M.; Dunbar, K. TRENDS in Microbiology, 2001, 9(3), 97-101; the entire contents of each of which are incorporated herein by reference). Any known sortase can be used as a starting enzyme in an evolution strategy provided herein, and the invention is not limited in this respect. For example, the present invention encompasses embodiments relating to a sortase A from any bacterial species or strain. The invention encompasses embodiments relating to a sortase B from any bacterial species or strain. The invention encompasses embodiments relating to a class C sortase from any bacterial species or strain. The invention also encompasses embodiments relating to a class D sortase from any bacterial species or strain. Amino acid sequences of sortases and the nucleotide sequences that encode them are known to those of skill in the art and are disclosed in a number of references cited herein, the entire contents of all of which are incorporated herein by reference. Those of skill in the art will appreciate that any sortase and any sortase recognition motif can be used in some embodiments of this invention, including, but not limited to, the sortases and sortase recognition motifs described in Ploegh et al., International PCT Patent Application, PCT/US2010/000274, filed Feb. 1, 2010, published as WO 2010/087994 on Aug. 5, 2010; Ploegh et al., International Patent Application PCT/US2011/033303, filed Apr. 20, 2011, published as WO 2011/133704 on Oct. 27, 2011; Liu et al., U.S. provisional Patent Application 61/662,606, filed on Jun. 21, 2012; and Liu et al., U.S. provisional Patent Application 61/880,515, filed on Sep. 20, 2013; the entire contents of each of which are incorporated herein by reference. The invention is not limited in this respect.

In some embodiments, the sortase is sortase A of S. aureus, or a variant thereof. The amino acid sequence of wild type sortase A of S. aureus is known to those of skill in the art, and a representative sequence (gi|21284177|ref|NP_{—}647265.1) is provided below:

MKKWTNRLMTIAGVVLILVAAYLFAKPHIDNYLHDKDKDEKIEQYDKNVK
EQASKDKKQQAKPQIPKDKSKVAGYIEIPDADIKEPVYPGPATPEQLNRG
VSFAEENESLDDQNISIAGHTFIDRPNYQFTNLKAAKKGSMVYFKVGNET
RKYKMTSIRDVKPTDVEVLDEQKGKDKQLTLITCDDYNEKTGVWEKRKIF
VATEVK (SEQ ID NO: 1, residues P94, D160, D165,
K190, and K196 are bold and underlined).

In some embodiments, a variant of sortase A is employed, e.g., a truncated, mutated, or tagged form of sortase A. Suitable variant forms of sortase A are described in more detail elsewhere herein, and include, for example, forms with a deletion of the N-terminal sequence KKWTNRLMTIAGVVLILVAAYLFAKPHIDNYLHDKDKDEKIEQYDKNVKEQASKDK KQ (SEQ ID NO: XX), or a part thereof; forms of sortase A comprising a tag, e.g., a histidine tag; and forms comprising a mutation, e.g., a mutation as described herein. One exemplary suitable form of sortase A is a sortase comprising the pentamutant sortase A sequence provided below:

(SEQ ID NO: 2)
MQAKPQIPKDKSKVAGYIEIPDADIKEPVYPGPATREQLNRGVSFAEEN
ESLDDQNISIAGHTFIDRPNYQFTNLKAAKKGSMVYFKVGNETRKYKMT
SIRNVKPTAVEVLDEQKGKDKQLTLITCDDYNEETGVWETRKIFVATEV
KLE.

Additional suitable S. aureus sortase A sequences will be apparent to those of skill in the art and the invention is not limited in this respect. In some embodiments, the sortase is a sortase A of another organism, for example, from another bacterial strain, such as S. pyogenes. In some embodiments, the sortase is a sortase B, a sortase C, or a sortase D. Suitable sortases from other bacterial strains will be apparent to those of skill in the art, and the invention is not limited in this respect.

The term “sortase substrate,” as used herein refers to a molecule or entity that can be utilized in a sortase-mediated transpeptidation reaction. Typically, a sortase utilizes two substrates—a substrate comprising a C-terminal sortase recognition motif, and a second substrate comprising an N-terminal sortase recognition motif and the transpeptidation reaction results in a conjugation of both substrates via a covalent bond. In some embodiments the C-terminal and N-terminal recognition motif are comprised in the same protein, e.g., in the same amino acid sequence. Sortase-mediated conjugation of the substrates in such cases results in the formation of an intramolecular bond, e.g., a circularization of a single amino acid sequence, or, if multiple polypeptides of a protein complex are involved, the formation of an intra-complex bond. In some embodiments, the C-terminal and N-terminal recognition motifs are comprised in different amino acid sequences, for example, in separate proteins. Some sortase recognition motifs are described herein and additional suitable sortase recognition motifs are well known to those of skill in the art. For example, sortase A of S. aureus recognizes and utilizes a C-terminal LPXT motif and an N-terminal GGG motif in transpeptidation reactions. Additional sortase recognition motifs will be apparent to those of skill in the art, and the invention is not limited in this respect. A sortase substrate may comprise additional moieties or entities apart from the peptidic sortase recognition motif. For example, a sortase substrate may comprise an LPXT motif, the N-terminus of which is conjugated to any agent, e.g., a peptide or protein, a small molecule, a binding agent, a lipid, a carbohydrate, or a detectable label. Similarly, a sortase substrate may comprise a GGG motif, the C-terminus of which is conjugated to any agent, e.g., a peptide or protein, a small molecule, a binding agent, a lipid, a carbohydrate, or a detectable label. Accordingly, sortase substrates are not limited to proteins or peptides but include any moiety or entity conjugated to a sortase recognition motif.

The term “subject,” as used herein, refers to an individual organism. In some embodiments, is a mammal, for example, a human, a non-human primate, a baboon, a mouse, a rat, a cat, a dog, a cattle, a goat, a pig, or a sheep. In some embodiments, the subject is a vertebrate.

The term “surface,” as used herein in the context of materials, e.g., of solid support materials, refers to the exterior boundary or the outermost layer of a material. A surface typically constitutes the interface of a material with a different material and/or a different phase. For example, a surface may be the exterior boundary of a solid material comprised in an implanted medical device, e.g., of a solid polymer or copolymer, that is in contact with a liquid material of the host organism, e.g., with a body fluid, such as blood, serum, lymph, interstitial fluid, or cerebrospinal fluid.

### DETAILED DESCRIPTION OF CERTAIN EMBODIMENTS OF THE INVENTION

Functional molecules immobilized on surfaces, e.g., on solid support surfaces, allow one to confer enhanced or new functionalities onto such surfaces. Surface engineering with functional molecules has found widespread applications in many fields, including, for example, in biotechnology and medicine, medical devices, industrial catalysis, process engineering, food processing and packaging, and surface preservation against biofouling. One problem with functional molecules that are exposed to physiological conditions, environmental conditions, or conditions of industrial catalysis, is their decay over time, e.g., as a result of oxidative stress, changes in pH or temperature, or as a result of enzymatic activity. This decay is associated with a loss of molecular function and, in turn, with a limit on the useful life of functionalized surfaces. While in some situations, a functionalized surface can easily be replaced, e.g., by changing a resin on a binding column; in other fields, for example, with respect to implantable medical devices, surface replacement is not feasible and would likely require removal and re-implantation of the device. In some situations, loss of function of a modified surface may lead to catastrophic failure, e.g., blood coagulation triggered by an implanted device, such as an arterial catheter or stent. In other applications, it would be desirable to only exchange functional molecules that have decayed or have lost their activity on a functionalized surface, instead of having to replace the entire surface.

Current techniques to covalently modify surfaces with bioactive compounds have largely involved bioconjugate techniques that link nucleophilic motifs such as amines, thiols, and hydroxyls to their partner electrophiles. Abundant presentation of these motifs in the complex chemical landscape of biological systems reduces the efficiency of targeting payloads for regenerating these device surfaces in physiological environments. Recent advances in rapid and highly bioorthogonal chemistries, notably Staudinger ligation and copper-free click cycloaddition, have facilitated coupling of azide-tagged targets in living systems, but are not reversible and prohibit removal of the initial surface assembly that has been degraded or depleted of bioactivity.

While conventional surface-engineering chemistries do not allow for the reversible attachment of functionalized molecules, some aspects of this disclosure provide compositions, reagents, kits, and methods that allow for reversible, covalent conjugation of functionalized molecules to a surface. The technology disclosed herein can be employed to strip functional molecules from the surface in vivo or in situ and replace them with fresh functional molecules of the same type in vivo or in situ, thus allowing for periodic restoration of surface functionality to counteract loss of function due to molecular degradation and/or decay. For example, in the context of an implanted medical device, the technology disclosed herein allows for the periodic restoration of surface functionality, which increases the useful life span of the medical device. The technology described herein can also be employed to strip functional molecules from a surface and replace them with functional molecules of a different type, thus allowing for sequential changes in surface functionality.

In some embodiments, the technology described herein can be employed to attach functional molecules to a surface in physiological conditions, allowing for surface functionalization in vivo, e.g., on an implanted medical device, such as a catheter, vascular graft, cardiac valve, or stent. The disclosure also provides reagents, compositions, kits, and methods for the sortase-mediated release of immobilized functional molecules from a surface. This immobilization and the release can be effected in vitro, in vivo, or in situ. Sortase-mediated immobilization and release of functional molecules to and from a surface, as described herein, can be used to strip degraded and/or decayed functional molecules from a surface and then “re-charge” the surface with fresh functional molecules.

The technology provided herein is inspired by the Staphylococcus aureus sortase A (SrtA), which catalyzes covalent transpeptidation of a specific C-terminal sortase recognition motif, LPXT, to a specific N-terminal sortase recognition motif, oligoglycine.

One area where the technology described herein is particularly useful is the field of medical diagnostics. Such assays often rely on the high specificity displayed by a binding agent, e.g., an antibody, to recognize and bind a diagnostic analyte, and generally involve immobilization of the binding agent on a solid support. Repeated or continuous use of the diagnostic devices comprising such solid supports requires breaking the binding agent-analyte bond, e.g., the antibody-antigen bond, to reuse the immobilized immunologic agent, or requires elimination of the antibody-antigen complex from the support and immobilization of fresh binding agent on the support. Most techniques to break the binding agent-analyte bond require low pH and high ionic strength, or the use of proteases to degrade the binding agent, e.g., the antibody. These harsh conditions will denature the immobilized reagents and affect long-term performance. Alternatively, elimination and regeneration of the immobilized reagents completely restores the sensing characteristics of the assay. Current techniques to achieve this “recharge” are highly inefficient, and sortase-catalyzed transpeptidation affords a rapid, specific, and reversible chemoenzymatic approach to replenish the functional characteristics of diagnostic devices.

Medical devices in blood contacting applications such as extracorporeal support systems, vascular access, and permanent implants are prone to life threatening complications initiated by maladaptive host biological responses at the blood-material interface. Immobilization of bioactive molecules and drug eluting assemblies on implantable devices has yielded promising combination products that mitigate thrombotic cascades and detrimental inflammation, enhance device integration and regeneration of healthy tissue, and inhibit microbial colonization. Clinical translation of these strategies for permanent implants has been constrained, in part, by the limited therapeutic duration afforded by a finite surface reservoir of bioactive agents, as well as degradation of surface components following exposure to the physiological environment. Efforts to improve biostability and bioactivity have included the manipulation of surface properties such as hydrophilicity, charge, and topography, immobilization chemistry, as well as rational and evolutionary protein engineering. Despite advances in these areas, a surface coating for implantable devices that reliably retains biological activity for a commercially and clinically viable time scale have not been developed. Some aspect of this disclosure provide compositions, reagents, kits, and methods for the restoration of medical device surface functionality by systemic delivery of functional molecules, e.g., bioactive therapeutic payloads, that are targeted to the respective device surfaces, e.g., blood-contacting surfaces, of a medical device, and effect in vivo or in situ regeneration of surface bioactivity. This concept of targeted, sortase-mediated immobilization of systemically administered functional molecules to a specific, engineered surface is applicable to fields other than the medical device field, e.g., to the field of targeted drug delivery, molecular imaging, and minimally invasive cell therapy.

The strategies for immobilizing functional molecules on surfaces, as well as for stripping and replacing such molecules on surfaces, employ sortase-mediated transpeptidation. Transpeptidation by sortase is suitable for immobilizing a broad range of functional molecules due to the synthetic simplicity of incorporating a sortase recognition motif, e.g., a C-terminal LPXT sequence or an N-terminal polyglycine sequence, on functional molecules. For example, functional proteins, such as thrombomodulin (TM), heparin, CD39, PEG, and other peptides can be fused to such a sortase recognition sequence, while other functional molecules, such as carbohydrates, lipids, or nucleic acids, can be conjugated to the peptidic sortase recognition motifs by methods known to those of skill in the art.

In addition, variants of WT SrtA or of 5′SrtA may be employed in the strategies for immobilizing and stripping functional molecules on solid surfaces described herein. In some embodiments, a SrtA with increased catalytic activity as compared to WT or 5′ SrtA is employed to improve the immobilizing and/or stripping efficiency. In some embodiments, SrtA with an altered substrate specificity is employed, e.g., Srt A that specifically recognizes LPESG, LAETG, or LAESG substrates, and can be used even in the presence of competing LPETG (SEQ ID NO: 4) peptide under physiological conditions, e.g, at 37° C. in serum. The use of such substrate-variant SrtA allows for the immobilization of two or more different functional molecules on the same surface, e.g., one with an LPXT sortase recognition motif and one with an LPXS recognition motif, and a specific, targeted release and/or re-charge of one of the immobilized molecules, but not the other(s), by contacting the surface with a sortase that specifically recognizes one of the sortase recognition motifs, but not the other(s). The use of sortase variants, accordingly, is useful in generating multimeric and/or heterofunctional bioenzyme assemblies, e.g., assemblies that synergize the activity of surface enzyme assemblies which resist platelet and coagulation activation cascades, as well as biofilm formation, to improve the durability of implantable medical devices that operate in contact with blood.

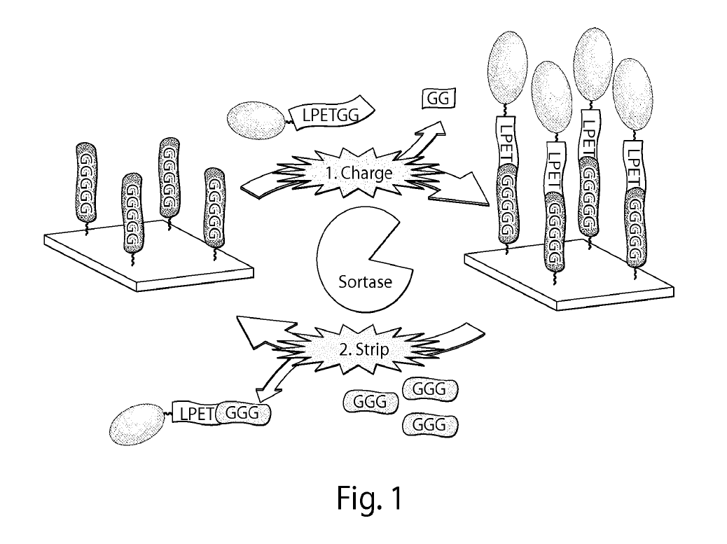

Some aspects of this disclosure provide for methods that involve immobilizing a functional molecule on a surface, e.g., immobilizing TM on a catheter surface, via sortase-mediated transpeptidation, e.g., as illustrated in FIG. 1. In some embodiments, a two-step “rechargeable” surface assembly cycle is provided, which is initiated by sortase-catalyzed immobilization, or “charging,” of LPXT-tagged functional molecules on polyglycine-modified surfaces, followed by “stripping” of the immobilized functional molecules by contacting the surface with sortase and an excess or free polyglycine peptides, to regenerate polyglycine anchor sites for additional “charge”-“strip” cycles.

Some aspects of this disclosure provide a method for functionalizing a solid support surface by conjugating a functional molecule on the surface. In some embodiments, the method involves providing a solid support surface to which a first sortase recognition motif is conjugated, providing a functional molecule conjugated to a second sortase recognition motif, which can partake in a sortase transpeptidation together with the first sortase recognition motif, and contacting the support with the functional molecule in the presence of a sortase. The result of such a method is a transpeptidation reaction that links the functional molecule to the solid support, and thus confers the functionality, e.g., an anti-coagulant activity, binding activity, or anti-fouling activity, to the surface of the solid support. In some embodiments, the contacting is in vitro. In other embodiments, however, the contacting is in vivo.

For example, in some embodiments, the solid support surface is a surface of a medical device that is implanted in a subject, such as a vascular graft, catheter, or stent. In some such embodiments, the sortase and the peptide are administered to the subject, either locally or systemically. Because the sortase-mediated transpeptidation reaction is highly specific, and because most mammalian cells do not express sortase recognition motifs on the surface of their cells, systemic administration of sortase and functional molecules conjugated to peptides comprising sortase recognition motifs in effective amounts to functionalize an implanted device surface is typically not toxic to a subject. Accordingly, the methods provided here allow for the functionalization of implantable medical devices, e.g., of catheters, stents, cardiac valves, vascular grafts, pumps, heart pacemakers, artificial joints, and cardioverter defibrillators.

In some embodiments, the functional molecule is a molecule that inhibits coagulation, an anti-fouling agent, or a therapeutic agent. In some embodiments, the functional molecule is thrombomodulin (TM), also known as CD141 or BDCA-3. Thrombomodulin proteins and fragments of such proteins having anti-thrombotic and anti-coagulant activity are well known to those of skill in the art. Some exemplary TM sequences useful in the context of this disclosure are provided below. It will be understood, however, that additional TM sequences having anti-coagulant or anti-thrombotic activity are also useful in the context of this disclosure. The disclosure is not limited in this respect.

>gi|4507483|ref|NP_000352.1| thrombomodulin
precursor [Homo sapiens]
(SEQ ID NO: XX)
MLGVLVLGALALAGLGFPAPAEPQPGGSQCVEHDCFALYPGPATFLNASQ
ICDGLRGHLMTVRSSVAADVISLLLNGDGGVGRRRLWIGLQLPPGCGDPK
RLGPLRGFQWVTGDNNTSYSRWARLDLNGAPLCGPLCVAVSAAEATVPSE
PIWEEQQCEVKADGFLCEFHFPATCRPLAVEPGAAAAAVSITYGTPFAAR
GADFQALPVGSSAAVAPLGLQLMCTAPPGAVQGHWAREAPGAWDCSVENG
GCEHACNAIPGAPRCQCPAGAALQADGRSCTASATQSCNDLCEHFCVPNP
DQPGSYSCMCETGYRLAADQHRCEDVDDCILEPSPCPQRCVNTQGGFECH
CYPNYDLVDGECVEPVDPCFRANCEYQCQPLNQTSYLCVCAEGFAPIPHE
PHRCQMFCNQTACPADCDPNTQASCECPEGYILDDGFICTDIDECENGGF
CSGVCHNLPGTFECICGPDSALARHIGTDCDSGKVDGGDSGSGEPPPSPT
PGSTLTPPAVGLVHSGLLIGISIASLCLVVALLALLCHLRKKQGAARAKM
EYKCAAPSKEVVLQHVRTERTPQRL
>gi|6678339|ref|NP_033404.1| thrombomodulin
precursor [Mus musculus]
(SEQ ID NO: XX)
MLGIFFLGVLAPASLGLSALAKLQPTGSQCVEHECFALFQGPATFLDASQ
ACQRLQGHLMTVRSSVAADVISLLLSQSSMDLGPWIGLQLPQGCDDPVHL
GPLRGFQWVTGDNHTSYSRWARPNDQTAPLCGPLCVTVSTATEAAPGEPA
WEEKPCETETQGFLCEFYFTASCRPLTVNTRDPEAAHISSTYNTPFGVSG
ADFQTLPVGSSAAVEPLGLELVCRAPPGTSEGHWAWEATGAWNCSVENGG
CEYLCNRSTNEPRCLCPRDMDLQADGRSCARPVVQSCNELCEHFCVSNAE
VPGSYSCMCETGYQLAADGHRCEDVDDCKQGPNPCPQLCVNTKGGFECFC
YDGYELVDGECVELLDPCFGSNCEFQCQPVSPTDYRCICAPGFAPKPDEP
HKCEMFCNETSCPADCDPNSPTVCECPEGFILDEGSVCTDIDECSQGECF
TSECRNFPGSYECICGPDTALAGQISKDCDPIPVREDTKEEEGSGEPPVS
PTPGSPTGPPSARPVHSGVLIGISIASLSLVVALLALLCHLRKKQGAARA
ELEYKCASSAKEVVLQHVRTDRTLQKF

In some embodiments, the first sortase recognition motif is a polyglycine motif, and the second sortase recognition motif is an LPXT motif, wherein X represents any amino acid. In other embodiments, the first sortase recognition motif is an LPXT motif, and the second sortase recognition motif is a polyglycine motif. In some embodiments, the polyglycine motif comprises two or more contiguous glycine residues. In some embodiments, the polyglycine motif comprises 2, 3, 4, 5, 6, 7, 8, 9, or 10 contiguous glycine residues. In some embodiments, the LPXT motif comprises an LPXTG motif, wherein X represents any amino acid. In some embodiments, the LPXT motif comprises an LPETG (SEQ ID NO: 4) motif.

Non-limiting examples of sortases that can be used in the disclosed methods are described herein and additional suitable sortases will be apparent to those of skill in the art. For example, in some embodiments, a sortase is employed that comprises an amino acid sequence that is at least 90% homologous to the amino acid sequence of wild-type S. aureus Sortase A provided as SEQ ID NO: 1 or a fragment thereof having sortase activity, e.g., a fragment comprising at least amino acids 61-206 of SEQ ID NO 1. In some embodiments, a mutant sortase is employed. Typically, the mutant sortase exhibits enhanced reaction kinetics as compared to wild type sortase, e.g., a higher reaction efficiency or a higher reaction rate. Mutant sortases that are suitable are described elsewhere herein, and include, for example, sortases comprising one or more mutations selected from the group consisting of P94S, P94R, E106G, F122Y, F154R, D160N, D165A, G174S, K190E, and K196T.

Some aspects of this disclosure provide a method of releasing a functional molecule conjugated to a solid support from the support via sortase-mediated transpeptidation. In such embodiments, the functional peptide is conjugated to the solid support via sortase-mediated transpeptidation, or via a peptidic linker forming [solid support]-[1^{st} sortase recognition motif]-[2^{nd} sortase recognition motif]-[functional molecule] structure. The release of the functional molecule can be effected in such methods by contacting the conjugated functional molecule with sortase and an excess of a free peptide comprising a sortase recognition motif, e.g., a peptide comprising or consisting of [sortase recognition motif 10, e.g., GGG, GGGG (SEQ ID NO: 7), or GGGGG (SEQ ID NO: 6), which results in the sortase reaction equilibrium being shifted towards release of the conjugated molecule and conjugation of the free peptide, or a part thereof, to the solid support (see, e.g., FIG. 1).

In some embodiments, the method for releasing a functional molecule from a solid support, which is also referred to herein as “stripping.” In some embodiments, the method comprises contacting a functional molecule, e.g., a thrombomodulin molecule, that is conjugated to a solid support, e.g., to the surface of an implanted vascular graft, via a peptidic linker comprising a first and a second sortase recognition motif, e.g., GGG-LPETG (SEQ ID NO: 5), with a sortase in the presence of a peptide comprising the first sortase recognition motif, e.g., a GGGGG (SEQ ID NO: 6) peptide, under conditions suitable for the sortase to catalyze a transpeptidation reaction. In some embodiments, the transpeptidation reaction releases the functional molecule from the solid support, and restores the first sortase recognition motif on the solid support. See, e.g., FIG. 1 for an illustration of one exemplary embodiment.

In some embodiments, the contacting is in vitro. In other embodiments, however, the contacting is in vivo. Accordingly, the methods provided herein allow for the stripping of functionalized molecules from solid supports of a wide variety of devices and in a wide variety of contexts, e.g., of the surfaces of lab ware, such as culture vessels, resins of analytical columns, or of implanted medical devices. For example, in some embodiments, the solid support surface is a surface of a medical device that is implanted in a subject, such as a vascular graft, catheter, or stent. In some such embodiments, the sortase and the peptide are administered to the subject, either locally or systemically. Because the sortase-mediated transpeptidation reaction is highly specific, and because most mammalian cells do not express sortase recognition motifs on the surface of their cells, systemic administration of sortase and peptides comprising sortase recognition motifs in effective amounts to functionalize an implanted device surface is typically not toxic to a subject. Accordingly, the methods provided here allow for the stripping of functional molecules from implantable medical devices, e.g., of catheters, stents, cardiac valves, vascular grafts, pumps, heart pacemakers, artificial joints, and cardioverter defibrillators. This is useful, for example, for the removal of decayed or damaged functional molecules from such surfaces, e.g., after a period of time in service, or when loss of functionality is detected or suspected.

The functionality of surfaces that have been stripped of functional molecules, e.g., according to a method provided herein, can be restored by re-functionalizing such surfaces with “fresh” functional molecules via sortase-mediated transpeptidation. The methods for re-functionalization are the same as those for initial functionalization in some embodiments, and may also use the same functional molecules. For example, in some embodiments, an implantable medical device surface may be functionalized with thrombomodulin, e.g., in vitro before implantation. The medical device may then be implanted into a subject, and, after some time in service, the surface may be stripped of the functionalized molecules, which may be damaged, decayed, or may otherwise have been rendered non-functional. Subsequently, the surface may be re-functionalized by conjugating “fresh” functional molecules to the surface. The stripping and re-functionalization can be effected in vivo without the need to explant the device, according to some embodiments. In some embodiments, a solid support surface to which functional molecules are conjugated via peptidic linkers comprising sortase recognition motifs as described herein can be stripped and re-functionalized (or “recharged”) at least 10 times. In some embodiments, the functional molecule that is stripped from the surface is different from the functional molecule that is used for recharging the surface. This allows, e.g., to use improved variants of functionalized molecules that are developed during the time of service of a device, e.g., enhanced thrombomodulin molecules. It also allows for changing the functionality of a surface according to the specific needs encountered. For example, in some embodiments, a surface may be functionalized with a binding agent, e.g., an agent that targets the device to a tissue or organ, then stripped, e.g., once it reached its destination, and then functionalized with a therapeutic agent, e.g., an agent that treats a disease of the tissue or organ.

Some aspects of this disclosure provide methods for inhibiting undesirable build-up of microorganisms on artificial surfaces, also known as “biofouling”, which can lead to catastrophic failure in permanently implantable medical devices and artificial organ systems, often requiring explantation and reimplantation of a new device. Biofouling also represents a problem in other fields of engineering, e.g., in a naval context, where the performance and useful life of the hulls of ships is currently severely limited by their lack of resistance to biofouling. The food-processing industry also benefits from rechargeable anti-microbial coatings for equipment surfaces that contact food. Some aspects of this disclosure provide methods for sortase-mediated immobilization of functional molecules having anti-fouling properties on surfaces in need of protection against biofouling. In some embodiments, the immobilization of such anti-fouling agents follows the methods for surface functionalization as described herein, with the functional molecule being an anti-fouling agent. The result is typically the generation of an anti-fouling film on the surface, which confers protection against biofouling for a period of time. The film can subsequently be “stripped” and “recharged” with fresh film on demand in order to achieve long-term protection.

Some aspects of this invention provide methods for the purification of a molecule of interest, e.g., an analyte or a product of a synthesis process. These methods are useful in the context of diagnostics and production of biological molecules, and in particular in the context of the purification of recombinant therapeutic proteins. Methods are provided herein that include the production of a recombinant protein of interest that comprises a sortase recognition motif, e.g., in the form of a C-terminal LPXT tag. In some embodiments, such recombinant proteins are produced by cells or in tissue culture. In some embodiments, the recombinant proteins are isolated from a sample, e.g., a sample comprising a crude lysate of the cells or culture, by immobilization and release of the recombinant proteins to a solid support via sortase-mediated transpeptidation, as described herein. In some embodiments, the solid support is a resin or a membrane, for example, a resin or membrane of a purification column or an analytical column. In some embodiments, the recombinant protein is immobilized on the solid support by contacting the solid support with a sample comprising the protein in the presence of a sortase and under conditions suitable for sortase-mediated transpeptidation. This results in immobilization of the recombinant protein on the solid support. Unbound components of the sample can then be washed away and the immobilized recombinant protein can be released from the column by stripping, e.g., by contacting the support with sortase and an excess of a peptide comprising a sortase recognition motif.

In some embodiments, a method for the generation of cell sheets in vitro is described that employs cell adhesion molecules that are immobilized on the surface of a culture vessel, e.g., a culture plate or culture dish via sortase-mediated transpeptidation. In some embodiments, cells are grown on the solid support and attach to the cell adhesion molecules. In some embodiments, the cells form cell sheets that are released from the cell culture vessel by contacting the cell culture vessel with a sortase and an excess of a free peptide comprising a sortase recognition motif, as described in more detail elsewhere herein. In some embodiments, sheets of cells with intact cell-cell contacts are obtained by such methods, which can be used in tissue engineering and transplantation.

In some embodiments, methods are provided for the functionalization of the surface of a cell. In some embodiments, such methods include expressing a sortase recognition motif on the surface of the cell, and then contacting the cell with a functional molecule conjugated to a sortase recognition motif in the presence of a sortase. In some embodiments, the functional molecule is a binding agent, or an agent protecting the cell from being targeted by the immune system of a host organism.

Some aspects of this invention employ sortases with enhanced reaction kinetics in order to improve the immobilization, stripping, and replacement efficiencies of the methods provided herein. In some embodiments, the enhanced sortase is an evolved sortase as described herein. In some embodiments, the evolved sortase exhibits enhanced reaction kinetics, for example, in that it catalyzes a transpeptidation reaction at a greater speed or turnover rate than the respective wild type sortase. In some embodiments, the enhanced sortase exhibits a modified substrate preference, for example, in that is utilizes a different substrate (e.g., a polypeptide comprising an altered sortase recognition motif) or binds a given substrate with higher or lower affinity, or with higher or lower specificity than the respective wild type sortase. In some embodiments, the sortase recognizes a sortase recognition motif that the respective wild type sortase does not recognize or bind.

For example, some embodiments of the present invention utilize a sortase comprising an amino acid sequence that is homologous to the amino acid sequence of a wild type sortase (e.g., to the amino acid sequence of S. aureus Sortase A as provided as SEQ ID NO: 1), or a fragment thereof. In some embodiments, the amino acid sequence of the utilized sortase comprises one or more mutations as compared to the wild type sequence of the respective sortase. For example, the evolved sortase sequence utilized may comprise 1, 2, 3, 4, 5, 6, 7, 8, 9, 10, 11, 12, 13, 14, 15, 16, 17, 18, 19, 20, 25, or more mutations. In some embodiments, the sequence of the utilized sortase is at least 90% identical, at least 95% identical, at least 98% identical, at least 99% identical, or at least 99.5% identical to a wild type sortase sequence.

In some embodiments, the evolved sortase is an evolved S. aureus sortase A. In some embodiments, the evolved sortase A comprises a mutation described herein, for example, a P94S, P94R, E106G, F122Y, F154R, D160N, D165A, G174S, K190E, or K196T mutation, or a combination of any of these mutations. In some embodiments, an evolved sortase is utilized that comprises 1, 2, 3, 4, 5, 6, 7, 8, or all 9 of these mutations. In some embodiments, an evolved sortase A is utilized that comprises a mutation that is homologous to the described mutations. For example, in some embodiments, an evolved sortase is utilized that comprises a P94S or P94R mutation, a D160N mutation, a D165A mutation, a K190E mutation, and a K196T mutation. In some embodiments, an evolved sortase is utilized that comprises a P94S or P94R mutation, a D160N mutation, and a K196T mutation. In some embodiments, an evolved sortase is utilized that comprises a P94S or P94R mutation, a D160N mutation, and a D165A mutation. In some embodiments, an evolved sortase is utilized that comprises a P94S or P94R mutation, a D160N mutation, a D165A mutation, and a K196T mutation. Such a pentamutant sortase A is sometimes referred to herein as “5′ SrtA.”

In some embodiments, an evolved sortase utilized in the present invention exhibits enhanced reaction kinetics, for example, in that it can achieve a greater maximum turnover per time unit (k_{cat}) or a greater turnover per time at physiological conditions. For example, in some embodiments, an evolved sortase is used according to the methods and strategies described herein that exhibits a k_{cat} that is at least 1.5-fold, at least 2-fold, at least 2.5-fold, at least 3-fold, at least 3.5-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, or at least 100-fold greater than the k_{cat} of the corresponding wild type sortase.

Some evolved sortases that are suitable for use in the context of surface modification exhibit enhanced reaction specificities, e.g., in that they bind a substrate with higher affinity or with higher selectivity, or in that they bind a substrate that is not bound or not efficiently bound by the respective wild type sortase. For example, some sortases utilized in the present invention exhibit a K_{M} for a substrate bound by the corresponding wild type sortase that is at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, or at least 50-fold less than the K_{M} of the corresponding wild type sortase for that substrate. Some evolved sortase A proteins useful in the present invention, for example, exhibit a K_{M} for a substrate comprising a C-terminal sortase recognition sequence of LPXT that is 2-fold, at least 3-fold, at least 4-fold, at least 5-fold, at least 6-fold, at least 7-fold, at least 8-fold, at least 9-fold, at least 10-fold less than the K_{M} of the corresponding wild type sortase A for a substrate comprising a C-terminal sortase recognition sequence of LPXT.

Some enhanced sortases useful in the present invention bind one of their substrates (e.g., a substrate with a C-terminal sortase recognition motif) with a decreased K_{M} while exhibiting no or only a slight decrease in the K_{M} for another substrate (e.g., a substrate with an N-terminal sortase recognition motif). For example, some evolved sortases exhibit a K_{M} for a substrate comprising a C-terminal sortase recognition motif (e.g., LPXT) that is at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, or at least 50-fold less than the K_{M} of the corresponding wild type sortase for that substrate, and also exhibit a K_{M} for a substrate comprising an N-terminal sortase recognition motif (e.g., GGG) that is not more than 2-fold, not more than 5-fold, not more than 10-fold, or not more than 20-fold greater than the K_{M} of the corresponding wild type sortase (e.g., wild type S. aureus sortase A).

In some embodiments, evolved sortases for use in the context of surface modification exhibit a ratio of K_{cat}/K_{M} for a substrate bound by the parent wild type sortase that is least 2-fold, at least 5-fold, at least 10-fold, at least 20-fold, at least 50-fold, at least 100-fold, or at least 120-fold greater than the K_{cat}/Kₘ ratio of the corresponding wild type sortase.

Some exemplary sortases suitable for use in the context of surface functionalization are described herein. Amino acid sequences of some additional, exemplary suitable sortases are described below. Changes relative to wild type S. aureus sortase A are underlined. Some sequences are truncated and/or comprise a C-terminal 6×His tag. Sequences comprising the deleted N-terminal sequence found in wild type Srt A (SEQ ID NO: 1), or any part of it, and sequences without the C-terminal 6×His tag are also functional and suitable for use according to some aspects of this disclosure.

wild type S. aureus sortase A (truncated, His
tagged)
(SEQ ID NO: 9)
MQAKPQIPKDKSKVAGYIEIPDADIKEPVYPGPATPEQLNRGVSFAEENE
SLDDQNISIAGHTFIDRPNYQFTNLKAAKKGSMVYFKVGNETRKYKMTSI
RDVKPTDVEVLDEQKGKDKQLTLITCDDYNEKTGVWEKRKIFVATEVKLE
HHHHHH
srtA P94S
(SEQ ID NO: 10)
MQAKPQIPKDKSKVAGYIEIPDADIKEPVYPGPATSEQLNRGVSFAEENE
SLDDQNISIAGHTFIDRPNYQFTNLKAAKKGSMVYFKVGNETRKYKMTSI
RDVKPTDVEVLDEQKGKDKQLTLITCDDYNEKTGVWEKRKIFVATEVKLE
HHHHHH
srtA D160N
(SEQ ID NO: 11)
MQAKPQIPKDKSKVAGYIEIPDADIKEPVYPGPATPEQLNRGVSFAEENE
SLDDQNISIAGHTFIDRPNYQFTNLKAAKKGSMVYFKVGNETRKYKMTSI
RNVKPTDVEVLDEQKGKDKQLTLITCDDYNEKTGVWEKRKIFVATEVKLE
HHHHHH
srtA D165A
(SEQ ID NO: 12)
MQAKPQIPKDKSKVAGYIEIPDADIKEPVYPGPATPEQLNRGVSFAEENE
SLDDQNISIAGHTFIDRPNYQFTNLKAAKKGSMVYFKVGNETRKYKMTSI
RDVKPTAVEVLDEQKGKDKQLTLITCDDYNEKTGVWEKRKIFVATEVKLE
HHHHHH
srtA K196T
(SEQ ID NO: 13)
MQAKPQIPKDKSKVAGYIEIPDADIKEPVYPGPATPEQLNRGVSFAEENE
SLDDQNISIAGHTFIDRPNYQFTNLKAAKKGSMVYFKVGNETRKYKMTSI
RDVKPTDVEVLDEQKGKDKQLTLITCDDYNEKTGVWETRKIFVATEVKLE
HHHHHH
Clone 4.2
(SEQ ID NO: 14)
MQAKPQIPKDKSKVAGYIEIPDADIKEPVYPGPATPEQLNRGVSFAEENE
SLDDQNISIAGHTFIDRPNYQFTNLKAAKKGSMVYFKVGNETRKYKMTSI
RNVKPTDVEVLDEQKGKDKQLTLITCDDYNEETGVWETRKIFVATEVKLE
HHHHHH
Clone 4.3
(SEQ ID NO: 15)
MQAKPQIPKDKSKVAGYIEIPDADIKEPVYPGPATSEQLNRGVSFAEENE
SLDDQNISIAGHTFIDRPNYQFTNLKAAKKGSMVYFKVGNETRKYKMTSI
RDVKPTAVEVLDEQKGKDKQLTLITCDDYNEKTGVWEKRKIFVATEVKLE
HHHHHH
P94S/D160N/D165A/K196T
(SEQ ID NO: 16)
MQAKPQIPKDKSKVAGYIEIPDADIKEPVYPGPATSEQLNRGVSFAEENE
SLDQDNISIAGHTFIDRPNYQFTNLKAAKKGSMVYFKVGNETRKYKMTSI
RNVKPTAVEVLDEQKGKDKQLTLITCDDYNEKTGVWETRKIFVATEVKLE
HHHHHH
P94S/D160N/K196T
(SEQ ID NO: 17)
MQAKPQIPKDKSKVAGYIEIPDADIKEPVYPGPATSEQLNRGVSFAEENE
SLDDQNISIAGHTFIDRPNYQFTNLKAAKKGSMVYFKVGNETRKYKMTSI
RNVKPTDVEVLDEQKGKDKQLTLITCDDYNEKTGVWETRKIFVATEVKLE
HHHHHH
P94S/D160N/D165A
(SEQ ID NO: 18)
MQAKPQIPKDKSKVAGYIEIPDADIKEPVYPGPATSEQLNRGVSFAEENE
SLDDQNISIAGHTFIDRPNYQFTNLKAAKKGSMVYFKVGNETRKYKMTSI
RNVKPTAVEVLDEQKGKDKQLTLITCDDYNEKTGVWEKRKIFVATEVKLE
HHHHHH
P94R/D160N/D165A/K190E/K196T
(SEQ ID NO: 19)
MQAKPQIPKDKSKVAGYIEIPDADIKEPVYPGPATREQLNRGVSFAEENE
SLDDQNISIAGHTFIDRPNYQFTNLKAAKKGSMVYFKVGNETRKYKMTSI
RNVKPTAVEVLDEQKGKDKQLTLITCDDYNEETGVWETRKIFVATEVKLE
HHHHHH

The disclosure contemplates use of wild-type and mutant Srt A enzymes. Some exemplary suitable wild type and mutant sortase enzymes are described in Liu et al., U.S. provisional Patent Application 61/662,606, filed on Jun. 21, 2012; and in Liu et al., U.S. provisional Patent Application 61/880,515, filed on Sep. 20, 2013; the entire contents of each of which are incorporated herein by reference. The reaction kinetics of wild type SrtA and some exemplary enhanced SrtA mutants useful in the context of the methods provided herein are summarized in Table 1. Each single mutation in isolation contributed a small beneficial effect on turnover (k_{cat}) and more significant beneficial effects on LPETG (SEQ ID NO: 4) substrate recognition, lowering the K_{m LPETG} up to threefold. The effects of the mutations in combination were largely additive. The tetramutant SrtA exhibited a 140-fold improvement in its ability to convert LPETG (SEQ ID NO: 4) (k_{cat}/K_{m LPETG}). This large gain in catalytic efficiency is achieved primarily through 45-fold improved LPETG (SEQ ID NO: 4) recognition accompanied by a 3-fold gain in k_{cat}.

The P94R/D160N/D165A/K190E/K196T pentamutant (5′ SrtA) is useful in embodiments that benefit from a high reaction efficiency. Compared to the tetramutant, the K_{m GGG} of this mutant improved by 1.8-fold, whereas the k_{cat} and K_{M LPETG} were not altered by more than a factor of 1.3-fold. Compared with wild type srtA, the pentamutant exhibits a 120-fold higher k_{cat}/K_{M LPETG} and a 20-fold higher K_{m GGG} (Table 1).

It will be apparent to those of skill in the art that the sortases and sortase variants described herein is detail are non-limiting examples, and that additional sortases and sortase variants can also be used in the context of the methods described herein. The disclosure is not limited in this respect.

TABLE 1
Kinetic characterization of wild type and mutant sortases:

| | | K_{m LPETG}, | k_{cat}/K_{m LPETG}, | K_{m GGG-COOH}, |
|---|---|---|---|---|
| | k_{cat}, s⁻¹ | mM | M⁻¹ s⁻¹ | μM |
| WT | 1.5 ± 0.2 | 7.6 ± 0.5 | 200 ± 30 | 140 ± 30 |
| D160N/K190E/ | 3.7 ± 0.6 | 1.6 ± 0.4 | 2,400 ± 700 | 1,200 ± 200 |
| K196T (clone 42) | | | | |
| P94S/D165A | 2.9 ± 0.0 | 1.1 ± 0.1 | 2,600 ± 100 | 1,700 ± 400 |
| (clone 4.3) | | | | |
| P94S/D160N/ | 4.8 ± 0.8 | 0.17 ± 0.03 | 28,000 ± 7,000 | 4,800 ± 700 |
| D165A/K196T | | | | |
| P94S/D1G0N/ | 4.8 ± 0.6 | 0.56 ± 0.07 | 8,600 ± 1,500 | 1,830 ± 330 |
| K196T | | | | |
| P94S/D160N/ | 3.8 ± 0.2 | 0.51 ± 0.38 | 7,500 ± 300 | 1,750 ± 250 |
| D165A | | | | |
| P94R/D160N/ | 5.4 ± 0.4 | 0.23 ± 0.02 | 23,000 ± | 2,900 ± 200 |
| D165A/ | | | 3,000 | |
| K190E/K196T | | | | |
| P94S | 1.6 ± 0.1 | 2.5 ± 0.6 | 600 ± 200 | 650 ± 150 |
| D160N | 2.3 ± 0.2 | 3.7 ± 0.5 | 600 ± 100 | 330 ± 20 |
| D165A | 2.4 ± 0.3 | 3.6 ± 1.0 | 700 ± 200 | 1,000 ± 480 |
| K196T | 1.2 ± 0.1 | 3.3 ± 0.8 | 400 ± 100 | 200 ± 70 |

Kinetic parameters k_{cat} and Kₘ were obtained from fitting initial reaction rates at 22.5° C. to the Michaelis-Menten equation. Errors represent the standard deviation of three independent experiments.

Additional suitable sortases include, without limitation, the following:

| Sortase | Mutations |
|---|---|
| 5mut: | P94R D160N D165A K190E K196T |
| 2A.3.5 | 5mut+ K162R V168I I182F |
| 2A.4 | 5mut+ A104H K162R V168I I182V |
| 2A.5 | 5mut+ R99H A104H K138I K162R I182V |
| 2A.6A | 5mut+ A104H K138V K162R I182V |
| 2A.6B | 5mut+ R99K A104H K138V D160K* K162R I182V |
| 2A.6C | 5mut+ A104H K138P K152I D160K* K162R I182V |
| 4S.3.5 | 5mut A104T A118T I182V |
| 4S.4 | 5mut+ A104V A118T F122S I182V |
| 4S.5 | 5mut+ N98D A104V A118T F122A K134R I182V |
| 4S.6A | 5mut+ N98D A104V A118S F122A K134G I182V E189V |
| 4S.6B | 5mut+ N98D A104V A118S F122A K134P I182V E189P |
| 4S.6C | 5mut+ N98D A104V A118T F122A K134R I182V E189F |

In some embodiments evolved sortases that efficiently use substrates not bound by the respective parent wild type sortase are employed. For example, in some embodiments, an evolved sortase is employed that is derived from a wild type S. aureus sortase A as the parent sortase A, which utilizes substrates comprising a C-terminal LPXT sortase recognition motif and substrates comprising an N-terminal GGG sortase recognition motif in a transpeptidation reaction. In some embodiments, the evolved sortases utilize a substrate different from those used by the parent sortase, e.g., substrates comprising a C-terminal LPXS, LAXT, LAXTG (SEQ ID NO: 20), MPXT, MPXTG (SEQ ID NO: 21), LAXS, LAXSG (SEQ ID NO: 22), NPXT, NPXTG (SEQ ID NO: 23), NAXT, NAXTG (SEQ ID NO: 24), NAXS, NAXSG (SEQ ID NO: 25), LPXP, LPXPG (SEQ ID NO: 26), or LPXTA (SEQ ID NO: 27) motif. In some embodiments, the evolved sortase comprises an S. aureus sortase A amino acid sequence, or a fragment thereof, with one or more of the following mutations: P86L, N98S, A104T, A118T, F122S, D124G, N127S, K134R, K173E, K177E and 1182V.

Those of skill in the art will understand that sortases recognizing any desired recognition motif can be used in the context of embodiments of this invention. It will be apparent to those of skill in the art that the engineered surface and the functional molecule have to include the respective N-terminal and C-terminal sortase recognition sequences in order for the substrate-modified sortase to effect transpeptidation. For example, a desired recognition motif may be longer or shorter than the corresponding wild type recognition motif, may comprise one or more amino acid substitutions, insertions, or deletions as compared to the corresponding wild type sortase recognition motif, or may be designed de novo, e.g., not based on any naturally occurring sortase recognition motif. The invention is not limited in this respect.

Some aspects of this invention provide methods for immobilizing a functional molecule on an engineered surface via sortase-mediated transpeptidation reactions, e.g., using the evolved sortases described herein. In some embodiments, such methods comprise contacting a sortase with a functional molecule, e.g., a protein comprising a suitable C-terminal sortase recognition motif, and an engineered surface comprising a suitable N-terminal sortase recognition motif under conditions suitable for sortase-mediated transpeptidation. In some embodiments, the evolved sortase is a sortase A, for example, an evolved S. aureus sortase A carrying one or more of the mutations described herein. In some embodiments, the C-terminal sortase recognition motif is LPXT, e.g., LPETG (SEQ ID NO: 4), and/or the N-terminal recognition motif is GGG.

The immobilizing transpeptidation reactions provided herein typically result in the creation of a protein fusion comprising the C-terminal sortase recognition motif and the N-terminal sortase recognition motif. In some embodiments, the functional molecule comprises a non-protein structure, e.g., a detectable label, a small molecule, a nucleic acid, a lipid, a polymer, or a polysaccharide. It will be apparent to those of skill in the art that the transpeptidation methods provided herein can be applied to conjugate any moieties that can be conjugated by any known sortase or sortase-mediated transpeptidation reaction, including, but not limited to, the reactions and moieties disclosed in Ploegh et al., International PCT Patent Application, PCT/US2010/000274, filed Feb. 1, 2010, published as WO 2010/087994 on Aug. 5, 2010; Ploegh et al., International Patent Application PCT/US2011/033303, filed Apr. 20, 2011, published as WO 2011/133704 on Oct. 27, 2011; Liu et al., U.S. provisional Patent Application 61/662,606, filed on Jun. 21, 2012; and in Liu et al., U.S. provisional Patent Application 61/880,515, filed on Sep. 20, 2013; the entire contents of each of which are incorporated herein by reference, for exemplary sortases, proteins, recognition motifs, reagents, moieties, and methods for sortase-mediated transpeptidation. The invention is not limited in this respect.

Some aspects of this invention provide methods for releasing a functional molecule immobilized on an engineered surface via a sortase-mediated transpeptidation reaction, e.g., using the evolved sortases described herein. In some embodiments, such methods comprise contacting a functional molecule immobilized on an engineered surface via a sortase-mediated transpeptidation reaction with a sortase, e.g., an evolved sortase described herein under conditions suitable for sortase-mediated release of the functional molecule from the surface. Such conditions typically comprise the presence of an excess of a free peptide comprising a sortase recognition motif, e.g., a polyglycine peptide, which shifts the equilibrium of the transpeptidation reaction. In some embodiments, the evolved sortase is a sortase A, for example, an evolved S. aureus sortase A carrying one or more of the mutations described herein. In some embodiments, the C-terminal sortase recognition motif comprises an LPXT motif, e.g., LPETG, and/or the N-terminal recognition motif comprises a GGG motif, and the free peptide comprises a GGG sequence, a GGGG (SEQ ID NO: 7) sequence, or a GGGGG (SEQ ID NO: 6) sequence.

The releasing transpeptidation reactions provided herein typically result in the release of a functional molecule comprising a C-terminal sortase recognition motif and a restoration of an N-terminal sortase recognition motif, e.g., GGG, on the surface.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1

### Rechargeable Surface Immobilization of Thrombomodulin to Generate a Sustainable, Biologically Active Blood Compatible Material

Polyurethane catheters modified with surface pentaglycine peptide motifs were generated and LPETG (SEQ ID NO: 4)-tagged thrombomodulin TM was immobilized on the catheter surfaces via sortase-mediated transpeptidation. Subsequently, 5′ SrtA was used to catalyze multiple cycles of rapid assembly and removal of LPETG (SEQ ID NO: 4)-tagged TM on pentaglycine-modified surfaces. Finally, this rechargeable surface engineering platform was translated to perform in vivo modification of catheters with LPETG (SEQ ID NO: 4) tagged functional TM molecules.

The development of clinically durable artificial organ systems has been limited, in part, by the activation of coagulation and platelets at the blood-material interface. Despite advances in surgical techniques and antithrombotic pharmaceutical therapies, there remains a need for synthetic small diameter (<6 mm) conduits that perform comparably to autologous grafts in the fields of cardiac, vascular, and plastic surgery. Central to the maladaptive thrombogenic process is surface-induced production of thrombin, which facilitates fibrin clot formation, activates platelets, and amplifies coagulation cascades through positive-feedback. Thrombomodulin is an endogenous attenuator of thrombin generation that directly sequesters thrombin, and catalyzes the activation of protein C which exerts negative feedback on the coagulation cascade. Through recombinant engineering of human TM and directed evolution of the bacterial transpeptidase sortase A (SrtA), the assembly of functional, biologically active TM was demonstrated on a range of surfaces. It is also shown herein that TM-modified surfaces reduce the thrombogenicity of commercial ePTFE grafts, and perform better than a commercially available heparin-coated Propaten grafts from Gore.

The bioactivity of functional molecules immobilized on surfaces interfacing with physiological environments, including TM and other biologically active surface coatings such as heparin, will decrease over time in vivo, thus compromising the functional characteristics of the surface modification. The lifetime of TM surface assemblies could be infinitely extended by “stripping” and “recharging” the surface using SrtA, as illustrated herein. The optimized rechargeable chemistry method described herein can be used in vitro and in vivo to facilitate regeneration of biologically active antithrombotic coating after deployment of modified devices.

Surfaces modified with TM can undergo at least 10×strip/recharge cycles in vitro without decrease in surface density.

In order to demonstrate the rechargeable surface assembly of biologically active enzymes, a C-terminal LPETG (SEQ ID NO: 4) tagged recombinant human thrombomodulin fragment (TM_{LPETG}) was generated. TM-mediated inhibition of blood coagulation represents a major physiological mechanism employed by the endothelial cell lining to attenuate activation of coagulation cascades in the arterial vasculature. Blood-contacting materials functionalized with TM exhibits lower thrombogenicity in vitro and in vivo. 5′ SrtA was generated by directed evolution using a yeast display system as described previously (see, Liu et al., U.S. provisional Patent Application 61/662,606, filed on Jun. 21, 2012; and in Liu et al., U.S. provisional Patent Application 61/880,515, filed on Sep. 20, 2013, the entire contents of which are incorporated herein by reference).

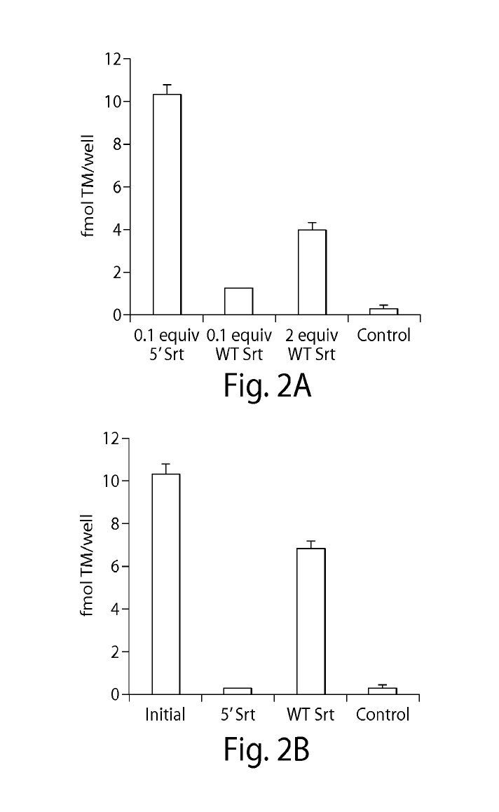

Model pentaglycine surfaces were generated by incubating NH₂-GGGGGK-biotin (SEQ ID NO: 8) peptide in 96-well microplates coated with streptavidin. Following surface immobilization with 1 μM TM_{LPETG} and 0.1 molar equivalents of either 5′ or WT SrtA in 20 mM Tris buffer (100 mM NaCl, 10 mM CaCl₂) for 1 hour, the quantity of immobilized TM was measured using a horseradish peroxidase conjugated TM antibody (FIG. 2A). 5′ SrtA was substantially more efficient in its capacity to immobilize TM_{LPETG} on pentaglycine surfaces compared with WT SrtA, as evident by the ˜9-fold higher surface TM density achieved using 5′ SrtA compared with WT SrtA under identical reaction conditions (FIG. 1A). Despite increasing the quantity of WT SrtA ˜20-fold higher than 5′ SrtA, final TM surface density yielded by WT SrtA only reached ˜40% of levels achieved by 5′ SrtA. Thus, compared with WT SrtA, 20-fold lower quantities of 5′ SrtA was required to achieve >2-fold greater quantities of TM_{LPETG}.

We also observed superior performance trends of 5′ SrtA compared with WT SrtA in stripping immobilized TM_{LPETG} from pentaglycine surfaces. In order to drive the detachment of TM_{LPETG}, high excess triglycine peptide (1 mM) was reacted with TM modified surfaces along with 0.1 molar equivalents 5′ SrtA with 20 μM 5′ or WT SrtA in 20 mM Tris buffer (100 mM NaCl, 10 mM CaCl2) for 30 minutes. 5′ SrtA-catalyzed stripping resulted in >90% removal of immobilized TM_{LPETG}, whereas WT SrtA was only able to remove ˜30% of the surface-bound TM under the same reaction conditions (FIG. 2B).

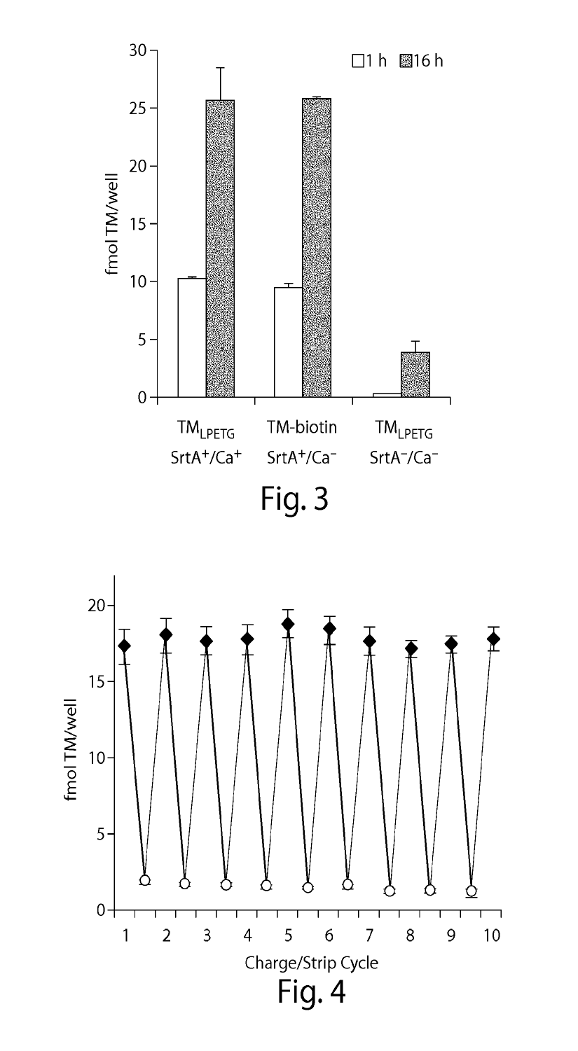

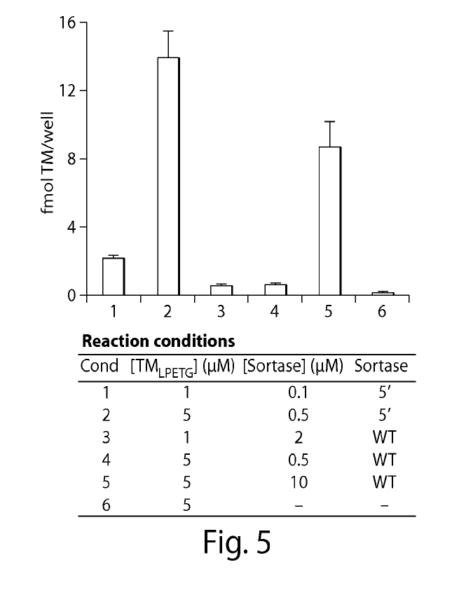

Further illustrating the rapid kinetic efficiency of 5′ SrtA-catalyzed TM_{LPETG} immobilization, the surface density of TM approached ˜50% of maximum surface density after 1 h reaction, and matched the immobilization efficiency achieved by biotin-streptavidin binding (FIG. 3). We further validate our concept that the recovery of surface pentaglycine anchor motifs, achieved by stripping the bound TM_{LPETG} using 5′ SrtA with excess triglycine, enabled further recharging using 5′ SrtA and fresh TM_{LPETG}. This two-step reaction achieved complete regeneration of the TM surfaces, even after 10 cycles (FIG. 4). Since biotin-streptavidin interaction is among the most widely used bioconjugate chemistry in life science research today, the robust bioorthogonal transpeptidation afforded by 5′ SrtA may be a viable alternative that achieves similar coupling efficiencies. In comparison with biotin-streptavidin binding, 5′ SrtA catalyzed transpeptidation is more reversible, requires less reagents, and facilitates tagging of substantially smaller nucleophile probes to target biomolecules.

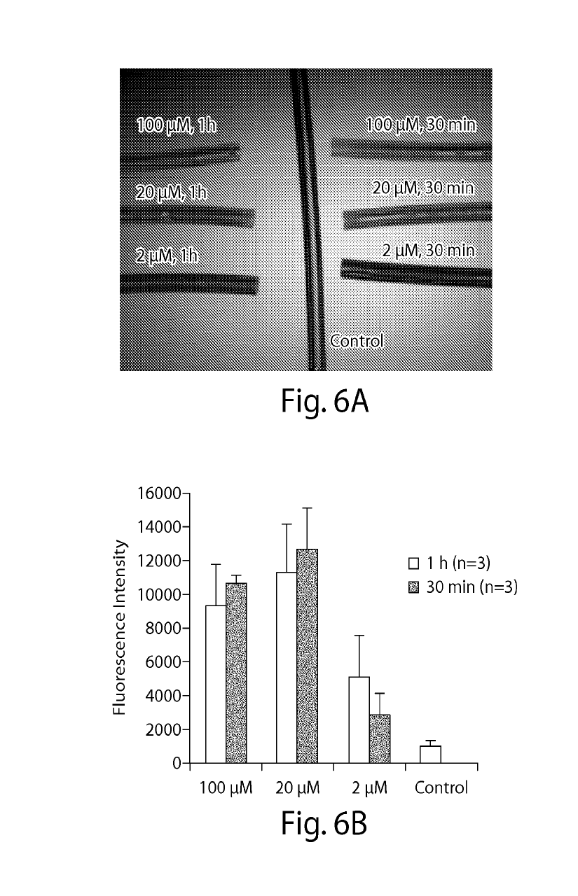

We next tested whether sortase-catalyzed transpeptidation could be carried out under a simulated in vivo environment. Freshly collected whole blood combined with heparin was diluted to 50% v/v with TM_{LPETG} and SrtA, and reacted for 1 h on pentaglycine modified surfaces at 37° C. without additional calcium. Under these conditions, 5′ SrtA-catalyzed transpeptidation required a 5-fold higher concentration of TM_{LPETG} to reproduce the surface densities achieved using the well-defined Tris buffer system. Although more TM_{LPETG} was required, 5′ SrtA-catalyzed immobilization yielded TM surface density levels that were about 20-fold higher than that achieved by WT SrtA (FIG. 5). Further increasing the quantity of WT SrtA 20-fold could only achieve ˜60% of the levels generated by 5′ SrtA. The surface modification reactions in whole blood highlight the impact of the 140-fold higher catalytic activity and specificity of 5′ SrtA relative to WT SrtA, and motivated further translation of our 2-step recharge scheme to modify blood contacting surfaces by systemic delivery of SrtA, TM_{LPETG}, and GGG formulations to the bloodstream in vivo.

### Example 2

### Sortase-Catalyzed Modification of Polyurethane Catheters

Polyurethane catheters intended for cannulation in mice were functionalized with pentaglycine using a sequential scheme as described previously to immobilize azide-tagged pentaglycine peptides. Optimal reaction parameters that would maximize SrtA-catalyzed charging and subsequent stripping of TM_{LPETG} on pentaglycine modified catheters were first determined ex vivo using a biotin-LPETG (SEQ ID NO: 4) probe. Fluorescent Cy3-labeled streptavidin was used to detect surface biotin, which was measured semi-quantitatively by image analysis.

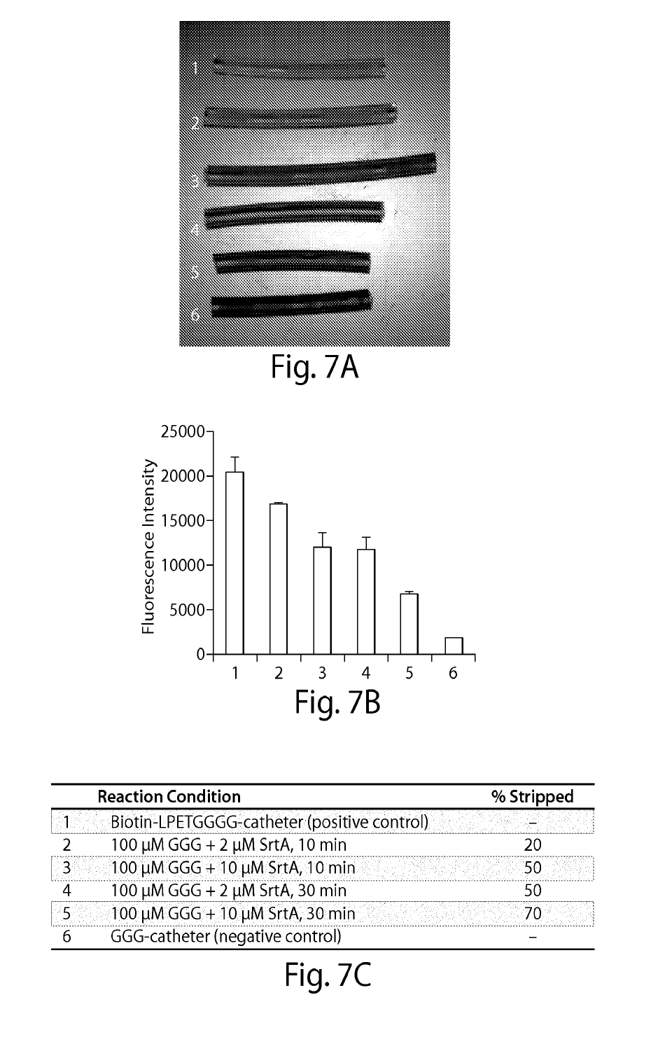

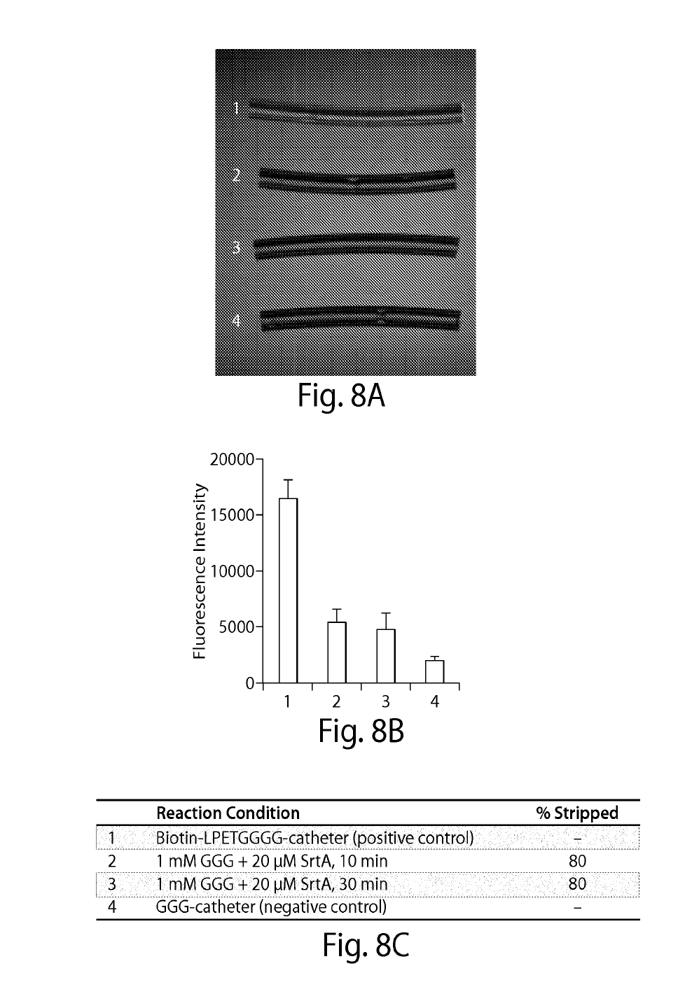

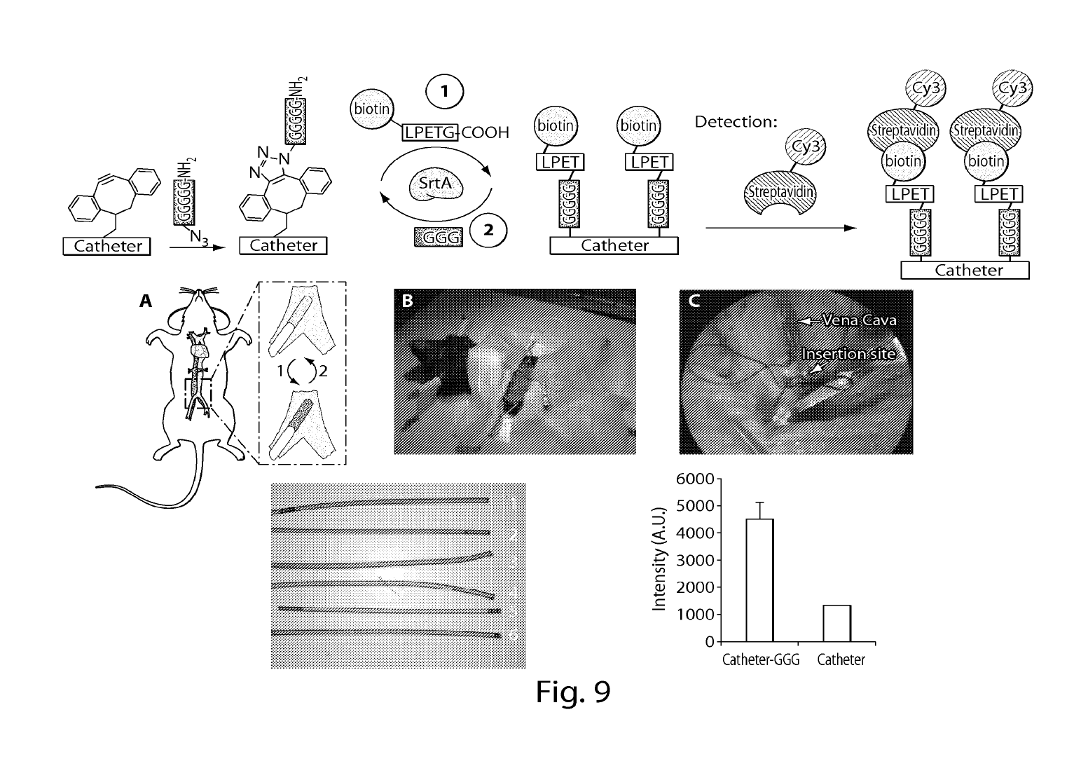

Under in vitro conditions, optimal charging reaction parameters were determined to be a 30 minute reaction with 20 μM biotin-LPETG (SEQ ID NO: 4) with 2 μM 5′ SrtA (FIG. 6), and stripping parameters were 1 mM GGG peptide with 20 μM 5′ SrtA (FIGS. 7 and 8). Similar to trends observed for TM_{LPETG} immobilization on model pentaglycine surfaces, a higher concentration of 5′ SrtA was required for removal of biotin-LPETG (SEQ ID NO: 4) than immobilization. Both WT SrtA and 5′ SrtA preferentially reacts with C-terminal LPETG (SEQ ID NO: 4) motifs, and therefore, the internal LPETG (SEQ ID NO: 4) motif of immobilized biotin-LPETG (SEQ ID NO: 4) and TM_{LPETG} was not an optimal recognition sequence for the stripping reaction. Additionally, the inherent reduction in degrees of freedom for surface reaction systems may have contributed to the lower efficiencies of SrtA-catalyzed stripping.

### Example 3

### Sortase-Catalyzed Rechargeable Surface Engineering In Vivo

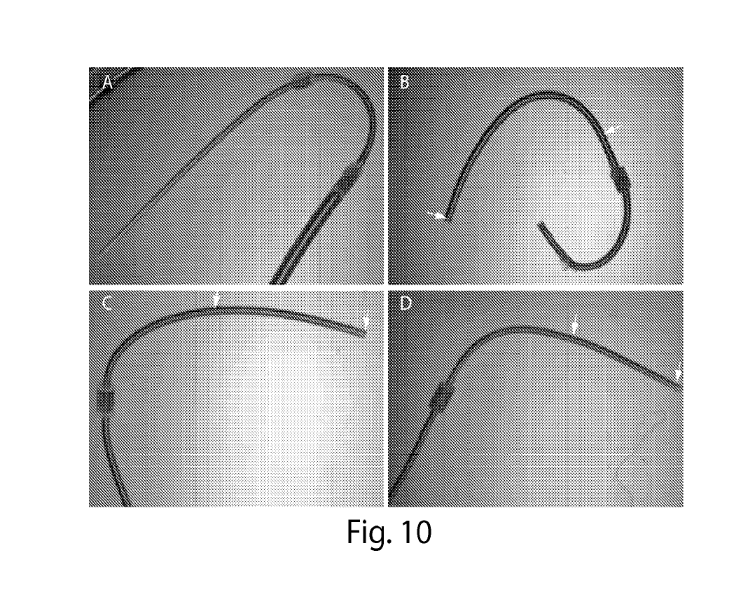

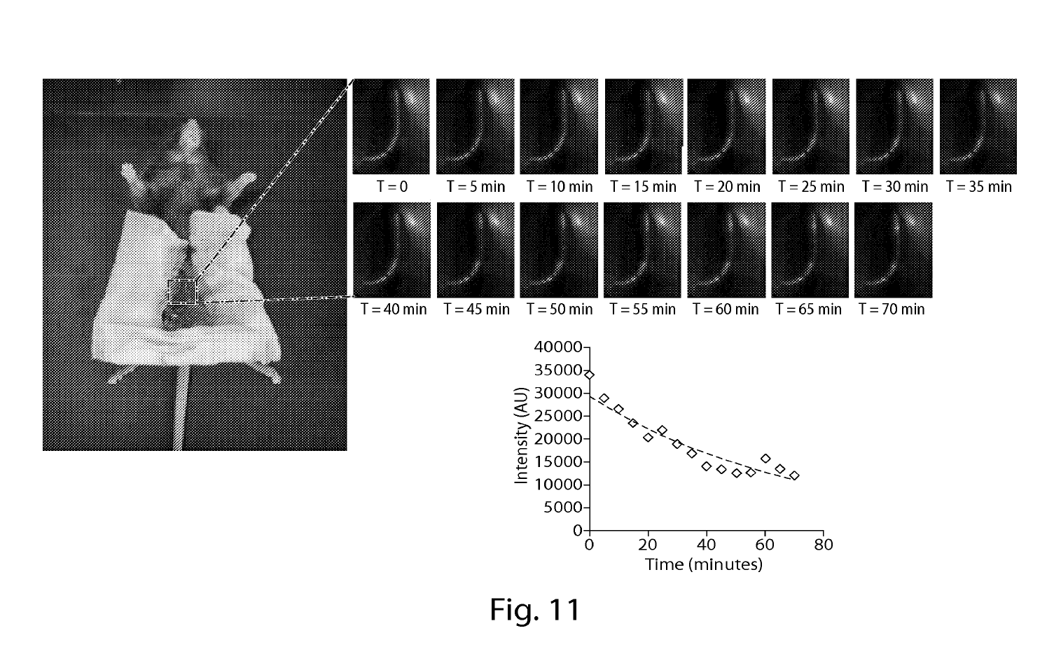

To demonstrate our concept that bioactive surface could be regenerated in vivo, we carried out reversible modification of pentaglycine-modified catheters with biotin-LPETG (SEQ ID NO: 4) probe in mice (FIG. 9). Pentaglycine catheters were cannulated through the femoral vein and deployed about 1 cm into the vena cava as measured from the bifurcation point. Next, biotin-LPETG (SEQ ID NO: 4) and 5′ SrtA were injected intravenously via the catheter, and after reaction for 30 minutes to 1 hour the catheters were removed. Fluorescent Cy3-labeled streptavidin was used to detect surface biotin on modified catheters. The in vivo charging of pentaglycine modified catheters could be achieved using a dose of 50 μg biotin-LPETG (SEQ ID NO: 4) and 70 μg 5′ SrtA after 30 minutes (FIG. 10). To confirm SrtA-catalyzed stripping of immobilized probes in vivo, catheters functionalized with biotin-LPETG (SEQ ID NO: 4) were deployed in the vena cava of mice. Immobilized biotin-LPETG (SEQ ID NO: 4) could be removed 1 hour following systemic administration of 400 μg triglycine peptide and 700 μg 5′ SrtA. Taken together, this evidence suggests that SrtA catalyzed regeneration of pentaglycine modified surfaces may be a viable in vivo surface engineering strategy.

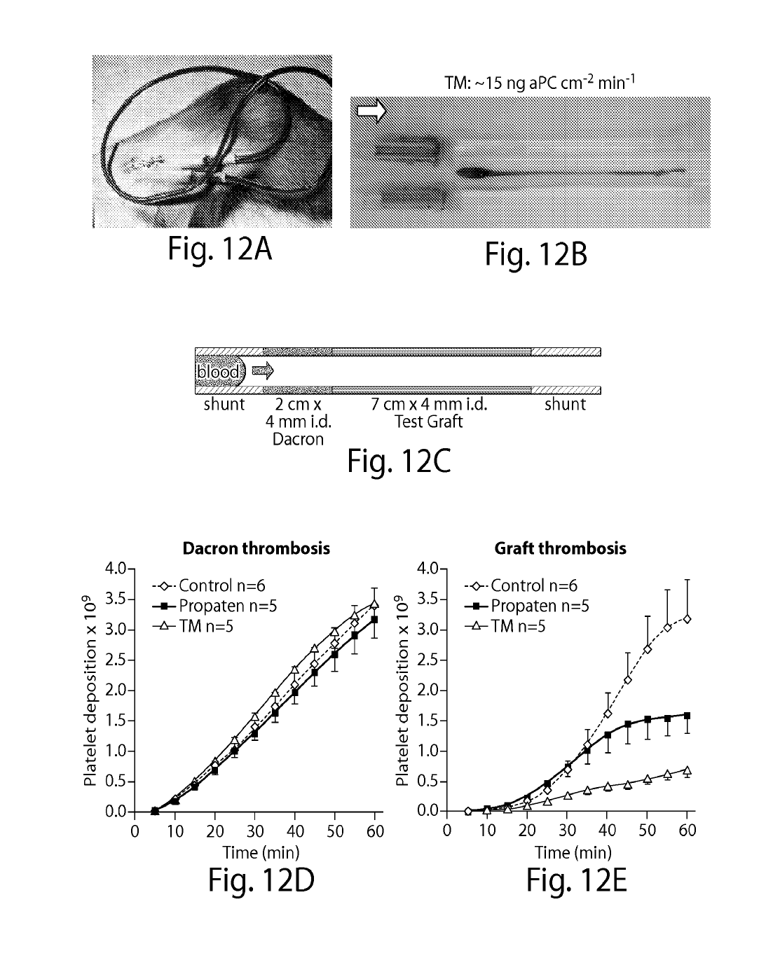

FIG. 11 shows real-time analysis of sortase-catalyzed stripping of catheters cannulated in a mouse vena cava.

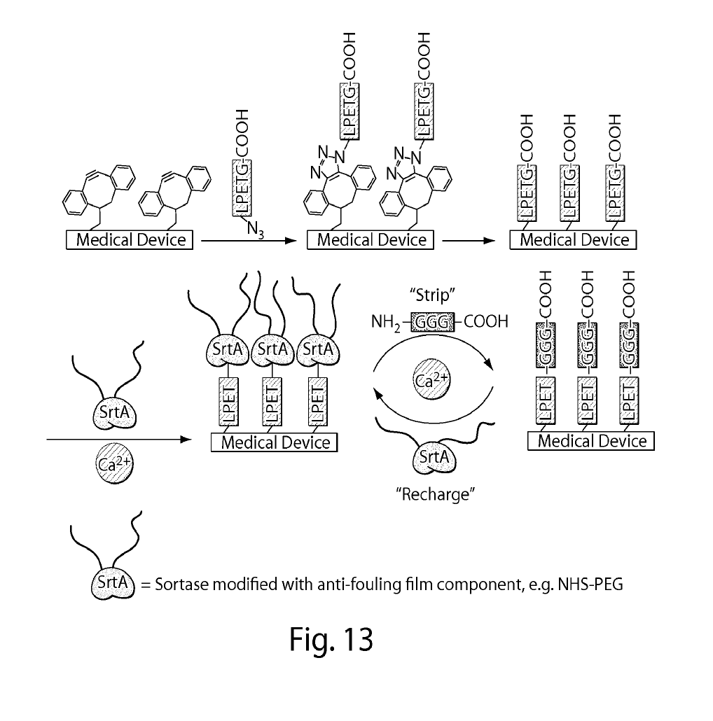

A baboon shunt model was used to evaluate the acute blood contacting properties of the TM-modified materials described herein and compare them to commercially available materials. FIG. 12A shows an exteriorized arteriovenous shunt model in non-human primates (baboon). Thrombosis was measured in a 2-chamber shunt configuration after 1 h perfusion in the shunt (FIG. 12B). A shunt configuration was used that employed a Dacron segment as a thrombotic stimulus upstream of test materials (FIG. 12C). Platelet deposition was measured in real time on the Dacron region and on either plain ePTFE control grafts from Gore, Propaten heparin coated grafts from Gore, and TM modified grafts (FIGS. 12D and E). The data show that TM-modified surfaces reduce the thrombogenicity of commercial ePTFE grafts, and perform better than a commercially available heparin-coated Propaten grafts from Gore.

### Example 4

### Rechargeable Anti-Fouling/Anti-Microbial Surface

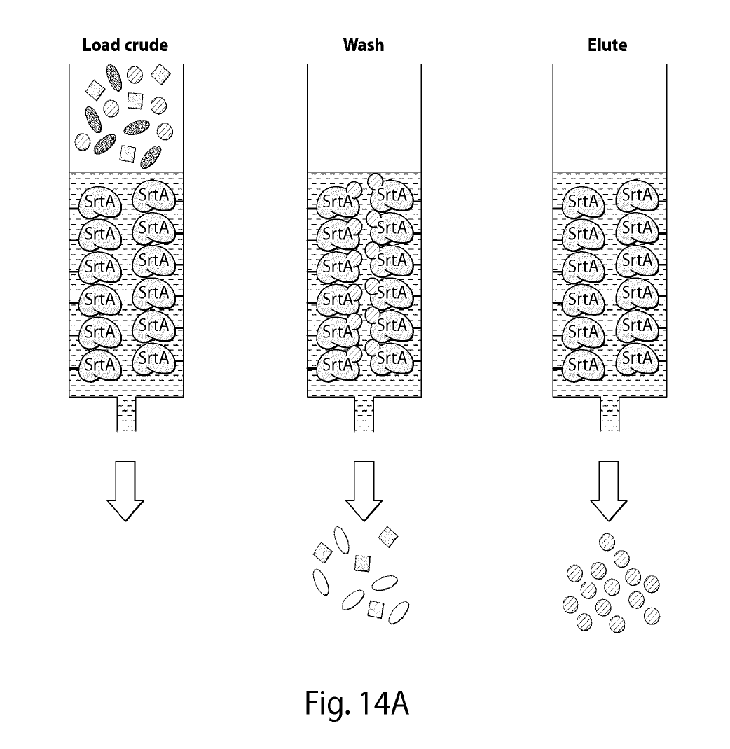

Undesirable build-up of microorganisms on artificial surfaces, also known as “biofouling”, can lead to catastrophic failure in permanently implantable medical devices and artificial organ systems, often requiring explantation and reimplantation of a new device. Biofouling also represents a problem in other fields of engineering, e.g., in a naval context, where the performance and useful life of the hulls of ships is currently severely limited by their lack of resistance to biofouling. The food-processing industry also benefits from rechargeable anti-microbial coatings for equipment surfaces that contact food. Current strategies to treat surface with active anti-microbial coatings or polymeric films to mitigate biofouling are limited by the finite lifetime of the coating, which often contain a reservoir of active reagents that are depleted over time. Sortase-mediated immobilization of functional molecules having anti-fouling properties is used to generate thin anti-fouling films on a range of solid supports, which can then be “stripped” and “recharged” with fresh film on demand in order to preserve the long-term functional characteristics of these films (FIG. 13).

The advantages of the sortase-mediated anti-fouling strategies provided herein include, but are not limited to, rapid stripping/recharge cycles that can be achieved with evolved sortases, the high specificity of stripping/recharging that allows for in situ recharging under virtually any environmental conditions where sortase is active, and the possibility of layer-by-layer assembly of polyelectrolytes, which can be used to assemble films of a range of thickness and functional characteristics.

### Example 5

### Protein Purification

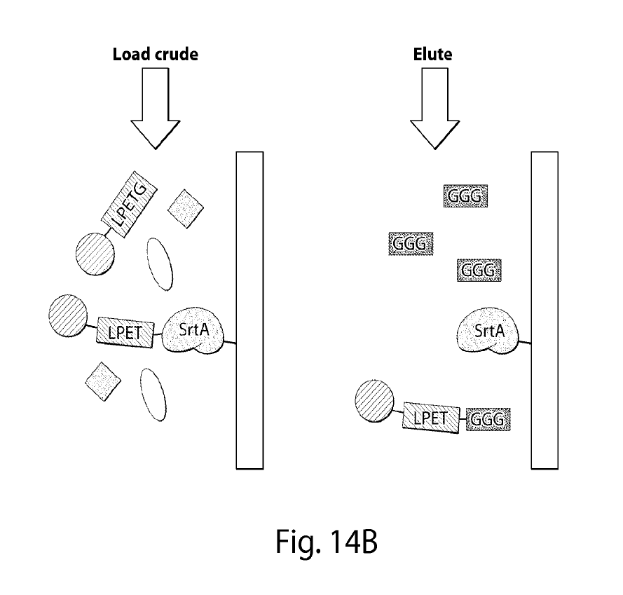

Many recombinant protein-based therapeutics (e.g. insulin, erythropoietin, therapeutic antibodies) require that the final product be free of undesirable contaminants. Affinity chromatography is the most powerful chromatographic method that maximizes recovery and purity of target recombinant protein from a crude mixture. Sortase-mediated immobilization and release as described herein is used to selectively anchor binding molecules, e.g., proteins of interest that contain the C-terminal LPXT sortase recognition motif, on a chromatography column. Such proteins can be released by contacting the column with sortase and a peptide comprising a sortase recognition motif, e.g., a GGG peptide. For example, a recombinant protein of interest can be engineered to carry a C-terminal LPXT tag. Such a protein can then be purified using a polyglycine column (FIG. 14).

Current affinity chromatography techniques that can be improved using sortase-mediated strategies described herein include, e.g., metal-chelate affinity (e.g., NTA-His tag), antibody affinity, glutathione affinity (e.g., binding glutathione S-transferase fusion proteins), avidin and streptavidin affinity (e.g., binding biotin tagged), lectin affinity (against sugar ligands) strategies. Advantages of using sortase-mediated strategies as described herein include, for example, a higher specificity than, e.g., metal-chelate affinity chromatography, which, in turn, leads to higher yields of pure LPXT-tagged proteins as compared to His-tagged proteins, lower costs in production of binding columns as compared to antibody or antibody fragment columns, a smaller footprint of the LPXT tag as compared to other tags (e.g., 6×His tag or GST tag (220 amino acid length)), faster off-kinetics rate compared to biotin-streptavidin, and a simple incorporation of a C-terminal LPXT motif by recombinant protein engineering, which is easier and more universal than the incorporation of other tags, e.g., GST tags, or sugar ligands for lectin affinity chromatography.

### Example 6

### Engineered Tissues for Regenerative Medicine

Development of regenerative medicine and tissue engineering require robust cell culture techniques. A critical step in the engineering of complex 3-D tissues is the generation of stable cellular sheets. A technology developed by Nishida et al. (N Engl J Med 2004) can be employed for the detachment of cell sheets without damaging their native cell-cell junctions. This technology has successfully been used to regenerate damaged cornea in the clinic, and is being studied to treat esophageal ulcerations and to repair damaged cardiac tissue. However, the cell sheet culture system requires a switch in temperature (37° C.-20° C.) to release the intact sheets that is not universally compatible with all cell types. Current cell detachment techniques such as trypsin digestion fail to generate intact sheets, as they disturb cell-cell junctions.

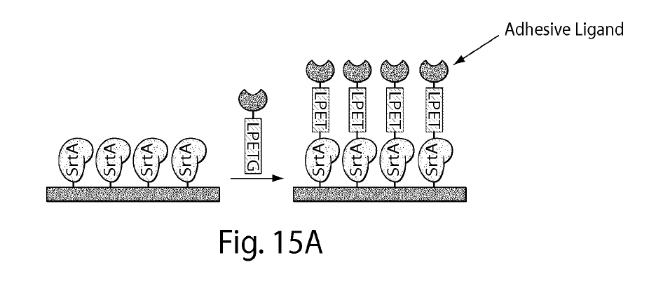

In some embodiments, sortase-mediated immobilization of cell adhesion ligands to cell culture vessels and sortase-mediated release of the ligands is used to produce cell sheets with intact cell-cell contacts, as illustrated in FIG. 15. Advantages of the sortase-catalyzed cell culture system provided herein include, but are not limited to, a cleavage of a chemical linkage between cell and vessel surface is used to release a cell sheet, which does not impact cell-cell contacts, the technology does not require changes in temperature for operation, and the methodology is universally applicable, as any LPXT tagged adhesion protein or polymeric film can be immobilized on a sortase coated dish.

### Example 7

### Evaluation of Sortase Variants

A number of sortase variants was developed by further mutating and evolving the pentamutant sortase and characterized:

TABLE 2
Kinetics data for evolved sortases. 5mut: Pentamutant sortase (having all five
mutations of the pentamutant). Additional mutations are listed for each respective sortase variant.

| Substrate/Target | wt/wt | 5mut/wt | 5mut/2A | 2A.3.5/2A | 2A.3.5/wt | 2A.4/2A |
|---|---|---|---|---|---|---|
| Mutations | — | P94R D160N | P94R D160N | 5mut + K162R | 5mut + K162R | 5mut + A104H |
| | | D165A K190E | D165A K190E | V168I I182F | V168I I182F | K162R V168I |
| | | K196T | K196T | | | I182V |
| kcat, 10 mM GGG | 1.220694008 | 5.621882686 | 1.315536327 | 1.248895247 | 1.586795141 | 1.900782516 |
| (Hz) | | | | | | |
| Sdev, kcat, 10 mM | 0.206127706 | 0.050996956 | 0.262907524 | 0.021650744 | 0.008262362 | 0.017265216 |
| GGG (Hz) | | | | | | |
| Km, LxExG (uM) | 5180.865735 | 207.8069987 | 6156.781755 | 7162.52724 | 4109.913889 | 3251.169918 |
| Sdev, Km, LxExG | 943.4564337 | 27.66474456 | 874.4527319 | 513.1042324 | 104.44959 | 113.3307861 |
| (uM) | | | | | | |
| kcat/Km (Hz/M) | 235.6158353 | 27053.38473 | 220.6162944 | 174.7051787 | 386.2215124 | 585.0234649 |
| Sdev, kcat/Km | 58.51434457 | 3609.890006 | 75.67625296 | 9.492630229 | 7.805955098 | 16.69296789 |
| (Hz/M) | | | | | | |
| specificity | | 122.6264126 | | 2.21070443 | | 73.04708671 |
| (relative cat. | | | | | | |
| Efficiency, fold) | | | | | | |
| Sdev, specificity | | 45.13405627 | | 0.128159767 | | 3.589253382 |
| (fold) | | | | | | |
| kHydrolysis (Hz) | 0.016574713 | 0.055463406 | | | | |
| Sdev, kHydrolysis | 0.008891304 | 0.040676362 | | | | |
| (Hz) | | | | | | |
| kcat, 1 mM LxExG | 0.17308818 | 7.245083652 | | | | |
| (Hz) | | | | | | |
| Sdev, kcat, 1 mM | 0.068138026 | 0.196190524 | | | | |
| LxExG (Hz) | | | | | | |
| Km, GGG (uM) | 290.6170509 | 2987.626804 | | | | |
| Sdev, Km, GGG | 221.9726779 | 80.92902606 | | | | |
| (uM) | | | | | | |
| Khydrolysis (uM) | 28.37924502 | 22.59305475 | | | | |
| Sdev, Khydrolysis | 21.646962 | 19.8961213 | | | | |
| (uM) | | | | | | |
| kL/KL/KH | 8.30E+06 | 1.20E+09 | | | | |
| Substrate/Target | 2A.4/wt | 2A.5/2A | 2A.5/wt | 2A.6A/2A | 2A.6A/wt | 2A.6B/2A |
| Mutations | 5mut + A104H | 5mut + R99H | 5mut + R99H | 5mut + A104H | 5mut + A104H | 5mut + R99K |
| | K162R V168I | A104H K138I | A104H K138I | K138V K162R | K138V K162R | A104H K138V |
| | I182V | K162R I182V | K162R I182V | I182V | I182V | D160K* |
| | | | | | | K162R I182V |
| kcat, 10 mM GGG | 0.093555041 | 1.409999086 | 0.137531365 | 2.469983633 | 0.15719454 | 0.69767657 |
| (Hz) | | | | | | |
| Sdev, kcat, 10 mM | 0.009667427 | 0.055318463 | 0.005753656 | 0.148040476 | 0.013824772 | 0.019749868 |
| GGG (Hz) | | | | | | |
| Km, LxExG (uM) | 11669.27262 | 433.3119304 | 4897.457635 | 608.3313756 | 3289.020619 | 309.9866552 |
| Sdev, Km, LxExG | 896.6218943 | 7.000387377 | 392.6284437 | 100.775363 | 298.3210472 | 28.84077154 |
| (uM) | | | | | | |
| kcat/Km (Hz/M) | 8.008854169 | 3255.914366 | 28.14079796 | 4128.180146 | 47.80226835 | 2266.706448 |
| Sdev, kcat/Km | 0.296497092 | 179.204205 | 1.096470124 | 646.4782722 | 0.58178654 | 261.6475808 |
| (Hz/M) | | | | | | |
| specificity | | 115.7008543 | | 86.35950319 | | |
| (relative cat. | | | | | | |
| Efficiency, fold) | | | | | | |
| Sdev, specificity | | 11.30238355 | | 5.406626636 | | |
| (fold) | | | | | | |
| kHydrolysis (Hz) | | 0.324012523 | | 0.225234539 | | 0.010481967 |
| Sdev, | | 0.025489666 | | 0.078893389 | | 0.002163477 |
| kHydrolysis (Hz) | | | | | | |
| kcat, 1 mM LxExG | | 1.492620267 | | 1.71404659 | | 0.535930039 |
| (Hz) | | | | | | |
| Sdev, kcat, 1 mM | | 0.07081996 | | 0.069056717 | | 0.031080138 |
| LxExG (Hz) | | | | | | |
| Km, GGG (uM) | | 2806.036691 | | 1039.788533 | | 642.2785789 |
| Sdev, Km, GGG | | 366.9256799 | | 237.2398425 | | 25.59263171 |
| (uM) | | | | | | |
| Khydrolysis (uM) | | 615.8786441 | | 135.7509813 | | 12.49068866 |
| Sdev, | | 140.4338096 | | 60.22602109 | | 2.764212493 |
| Khydrolysis (uM) | | | | | | |
| kL/KL/KH | | 5.29E+06 | | 3.04E+07 | | 1.81E+08 |
| Substrate/Target | 2A.6B/wt | 2A.6C/2A | 2A.6C/wt | 5mut/4S | 4S.3.5/4S | 4S.3.5/wt |
| Mutations | 5mut + R99K | 5mut + A104H | 5mut + | P94R D160N | 5mut A104T | 5mut A104T |
| | A104H | K138P K152I | A104H | D165A K190E | A118T I182V | A118T I182V |
| | K138V | D160K* | K138P K152I | K196T | | |
| | D160K* | K162R I182V | D160K* | | | |
| | K162R I182V | | K162R I182V | | | |
| kcat, 10 mM GGG | | 0.812860117 | 0.07675539 | 1.739673591 | 1.118147686 | |
| (Hz) | | | | | | |
| Sdev, kcat, 10 mM | | 0.09198838 | 0.002728785 | 0.174911912 | 0.164087961 | |
| GGG (Hz) | | | | | | |
| Km, LxExG (uM) | | 343.4888498 | 1983.065489 | 671.940841 | 7310.434447 | |
| Sdev, Km, LxExG | | 24.03918885 | 77.67622015 | 167.1750208 | 1308.709174 | |
| (uM) | | | | | | |
| kcat/Km (Hz/M) | | 2384.915833 | 38.70920459 | 2654.656128 | 153.8703055 | |
| Sdev, kcat/Km | | 428.6545792 | 0.191909734 | 436.5540965 | 12.72468908 | |
| (Hz/M) | | | | | | |
| specificity (relative | | 61.61107824 | | 10.19091868 | | |
| cat. Efficiency, | | | | | | |
| fold) | | | | | | |
| Sdev, specificity | | 11.07792379 | | 2.158175931 | | |
| (fold) | | | | | | |
| kHydrolysis (Hz) | | 0.010481967 | | | | |
| Sdev, kHydrolysis | | 0.002163477 | | | | |
| (Hz) | | | | | | |
| kcat, 1 mM LxExG | | 0.535930039 | | | | |
| (Hz) | | | | | | |
| Sdev, kcat, 1 mM | | 0.031080138 | | | | 8.841950988 |
| LxExG (Hz) | | | | | | |
| Km, GGG (uM) | | 642.2785789 | | | | |
| Sdev, Km, GGG | | 25.59263171 | | | | |
| (uM) | | | | | | |
| Khydrolysis (uM) | | 12.49068866 | | | | |
| Sdev, Khydrolysis | | 2.764212493 | | | | |
| (uM) | | | | | | |
| kL/KL/KH | | 1.91E+08 | | | | |
| Substrate/Target | 4S.4/4S | 4S.4/wt | 4S.5/4S | 4S.5/wt | 4S.6A/4S | 4S.6A/wt |
| Mutations | 5mut + A104V | 5mut + A104V | 5mut + N98D | 5mut + N98D | 5mut + N98D | 5mut + N98D |
| | A118T F122S | A118T F122S | A104V A118T | A104V A118T | A104V A118T | A104V A118T |
| | I182V | I182V | F122A K134R | F122A K134R | F122A K134G | F122A K134G |
| | | | I182V | I182V | I182V E189V | I182V E189V |
| kcat, 10 mM GGG | 0.726925959 | 0.095714145 | 0.387725911 | 0.006148609 | 0.953745987 | 1.102146085 |
| (Hz) | | | | | | |
| Sdev, kcat, 10 mM | 0.040738822 | 0.006560006 | 0.023554298 | 0.000608698 | 0.095078853 | 0.045369351 |
| GGG (Hz) | | | | | | |
| Km, LxExG (uM) | 1408.091941 | 338.7530289 | 75.99463534 | 12.18687654 | 102.3903263 | 1294.3279 |
| Sdev, Km, LxExG | 103.4411288 | 35.89713184 | 8.975668999 | 1.361443909 | 10.64783321 | 110.7342418 |
| (uM) | | | | | | |
| kcat/Km (Hz/M) | 516.9021989 | 283.4849351 | 5129.750723 | 513.0401911 | 9394.140707 | 854.1552296 |
| Sdev, kcat/Km | 19.06475492 | 15.31339113 | 392.5617968 | 114.3429095 | 1448.405056 | 53.20038819 |
| (Hz/M) | | | | | | |
| specificity | 1.823385072 | | 9.998730727 | | 10.99816565 | |
| (relative cat. | | | | | | |
| Efficiency, fold) | | | | | | |
| Sdev, specificity | 0.24057593 | | 0.160364958 | | 12.6411151 | |
| (fold) | | | | | | |
| kHydrolysis (Hz) | | | 0.009550657 | | 0.066957275 | |
| Sdev, kHydrolysis | | | 0.000364915 | | 0.045974206 | |
| (Hz) | | | | | | |
| kcat, 1 mM LxExG | | | 1.34223901 | | 1.930863111 | |
| (Hz) | | | | | | |
| Sdev, kcat, 1 mM | | | 0.328144639 | | 0.13952882 | |
| LxExG (Hz) | | | | | | |
| Km, GGG (uM) | | | 26698.41922 | | 4946.346135 | |
| Sdev, Km, GGG | | | 6134.780703 | | 1121.104516 | |
| (uM) | | | | | | |
| Khydrolysis (uM) | | | 190.7665753 | | 179.292082 | |
| Sdev, Khydrolysis | | | 17.86055357 | | 148.1163037 | |
| (uM) | | | | | | |
| kL/KL/KH | | | 2.69E+07 | | 5.24E+07 | |

| Substrate/Target | 4S.6B/4S | 4S.6B/wt | 4S.6C/4S | 4S.6C/wt |
|---|---|---|---|---|
| Mutations | 5mut + N98D | 5mut + N98D | 5mut + N98D | 5mut + N98D |
| | A104V A118S | A104V A118S | A104V A118T | A104V A118T |
| | F122A K134P | F122A K134P | F122A K134R | F122A K134R |
| | I182V E189P | I182V E189P | I182V E189F | I182V E189F |
| kcat, 10 mM GGG (Hz) | 1.241539966 | 1.080724703 | 0.665737178 | 0.003663126 |
| Sdev, kcat, 10 mM GGG | 0.108271856 | 0.101425203 | 0.16708905 | 0.000119561 |
| (Hz) | | | | |
| Km, LxExG (uM) | 311.9338134 | 237.5991947 | 156.478353 | 16.05552448 |
| Sdev, Km, LxExG (uM) | 94.98617736 | 17.47052549 | 28.44546328 | 3.73862534 |
| kcat/Km (Hz/M) | 4139.211117 | 4547.346117 | 4349.070663 | 234.8365865 |
| Sdev, kcat/Km (Hz/M) | 788.9272214 | 219.0365622 | 1447.408578 | 43.35556701 |
| specificity (relative cat. | 0.91024765 | | 18.51956174 | |
| Efficiency, fold) | | | | |
| Sdev, specificity (fold) | 0.761697464 | | 0.102605403 | |
| kHydrolysis (Hz) | 0.016704884 | | 0.013989042 | |
| Sdev, kHydrolysis (Hz) | 0.001313081 | | 0.002355155 | |
| kcat, 1 mM LxExG (Hz) | 2.131849488 | | 1.502291868 | |
| Sdev, kcat, 1 mM LxExG | 0.114779707 | | 0.02193525 | |
| (Hz) | | | | |
| Km, GGG (uM) | 15730.32228 | | 35983.54434 | |
| Sdev, Km, GGG (uM) | 2130.787148 | | 1480.295766 | |
| Khydrolysis (uM) | 122.32836 | | 334.1419054 | |
| Sdev, Khydrolysis (uM) | 4.050583082 | | 63.1306735 | |
| kL/KL/KH | 3.38E+07 | | 1.30E+07 | |

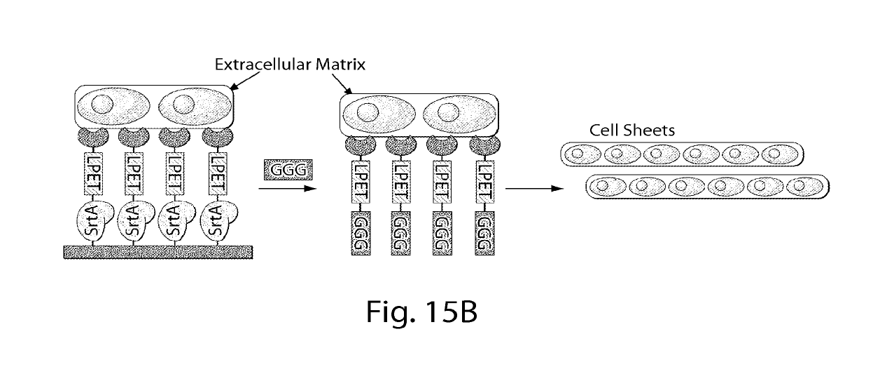

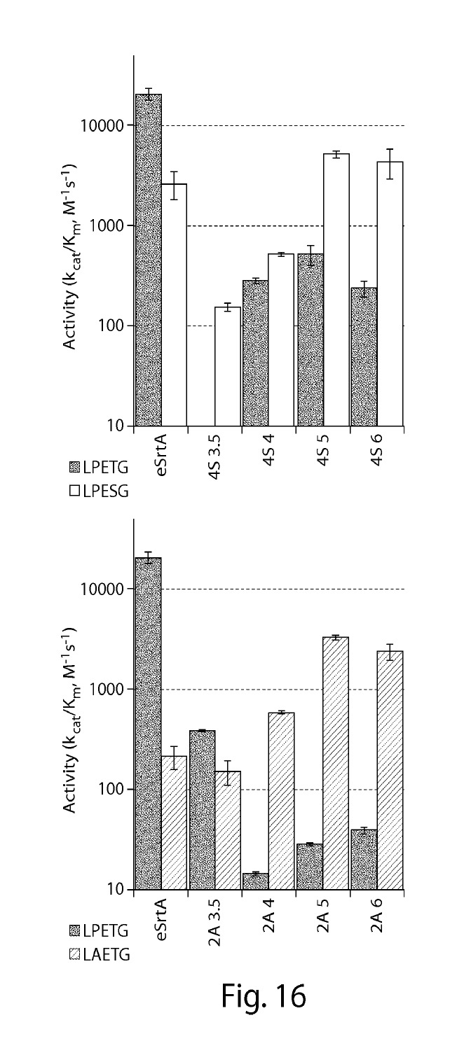

FIG. 16 shows a comparison of sortase activity on LPETG (SEQ ID NO: 4), LPESG, and LAETG motif-bearing substrates. Analysis of the sortase reaction kinetics revealed a fourth kinetic parameter (FIG. 17).

Analysis of K_{H} was performed in order to evaluate the characteristics of different sortase enzymes (Table 3):

TABLE 3

| SrtA Variant | k_{cat}/Kₘ (M⁻¹s⁻¹) | K_{Hydro} (μM) |
|---|---|---|
| Wild Type | 236 ± 59 | 14.3 ± 0.8 |
| Pentamutant | 27100 ± 3600 | 33.8 ± 1.9 |
| 2A.6c | 2380 ± 430 | 12.5 ± 2.8 |
| 4S.6c | 4350 ± 1400 | 334 ± 63 |

Above and beyond its increased catalytic rate, the pentamutant sortase showed about two times the hydrolysis rate of wild type sortase. Sortase 2A.6c showed hydrolytic performance exceeding that of the wild type enzyme, while sortase 4S.6c showed decreased hydrolytic performance (about 10% that of the pentamutant sortase).

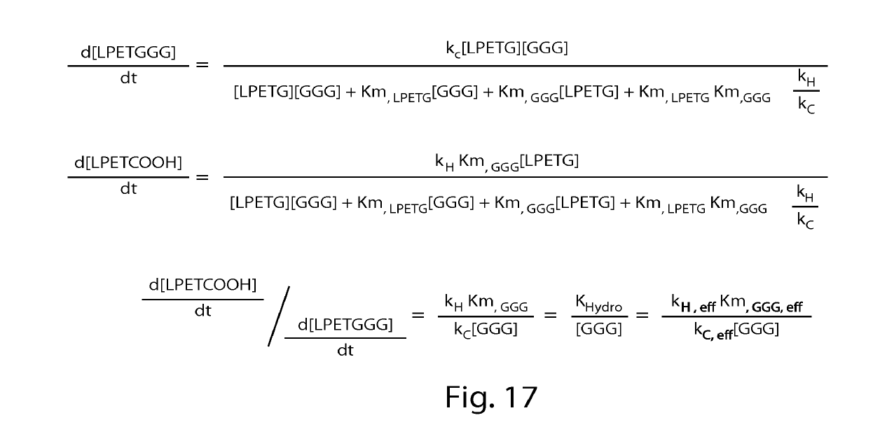

FIG. 18 shows the results of a sequence analysis of selected residues of evolved sortases (2A and 4S). For 2A sortases, the highest variability was observed in residues 164, 165, and 166, while for 4S sortases, the highest variability was observed in residues 118 and 122.

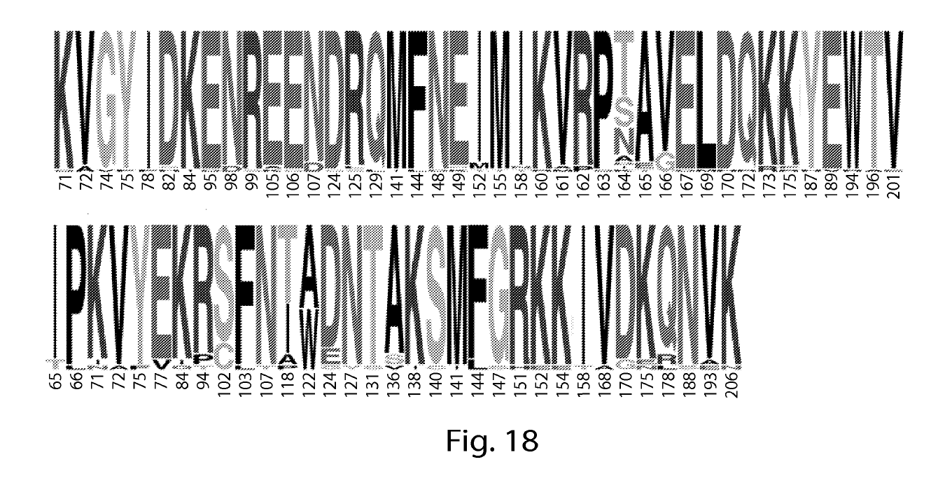

FIG. 19 shows structural models of some exemplary evolved sortases.

### Example 8

### Tissue Labeling with Different Sortase Variants

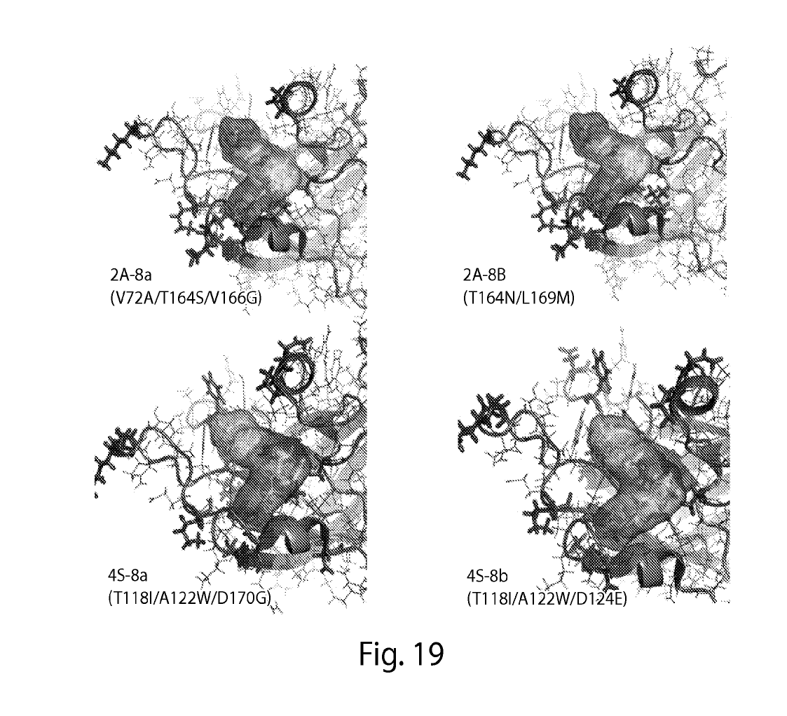

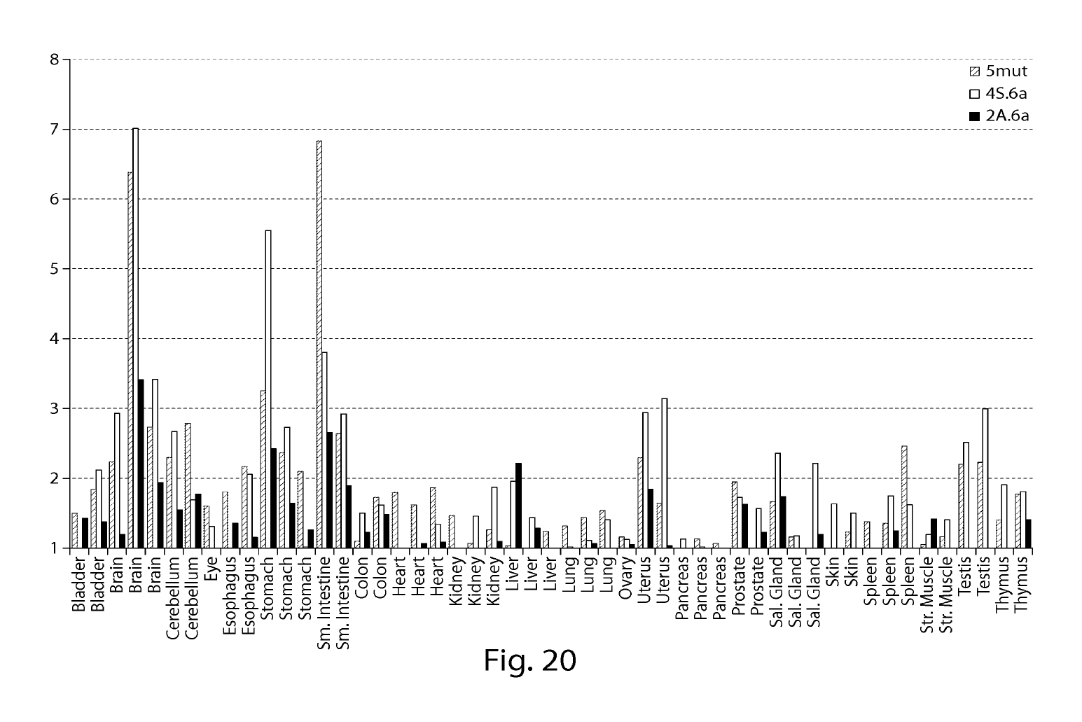

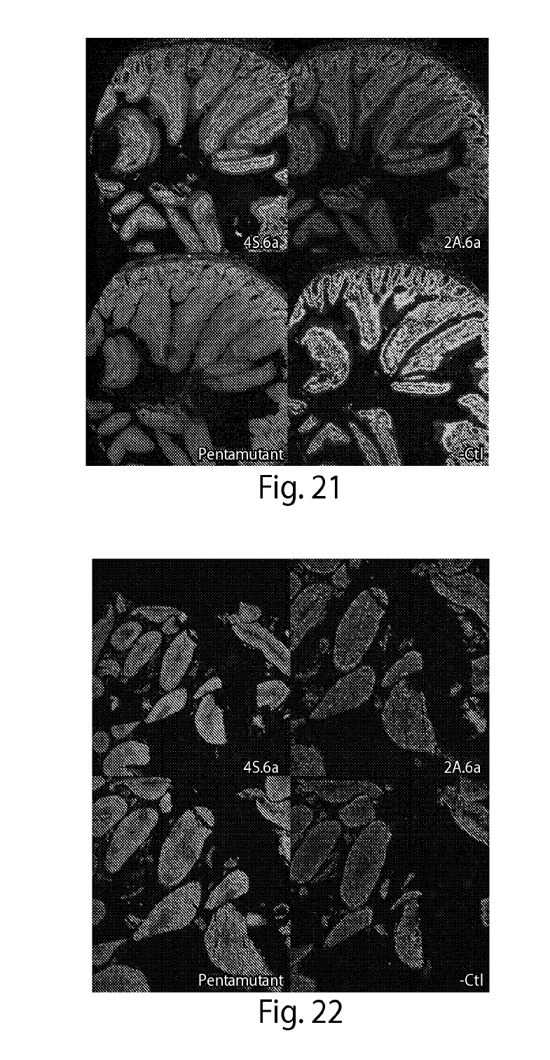

In order to evaluate the efficiency of on-slide tissue labeling, various tissues were treated with three different sortases (pentamutant, 4S.6a, and 2A,6a) and a detectable label comprising a sortase recognition motif. FIG. 20 shows an overview of labeling efficiencies observed in different tissues. FIGS. 21 and 22 show fluorescent micrographs of sortase-labeled small intestine and testis tissues.

### Example 9

### Sortase-mediated Protein Modification

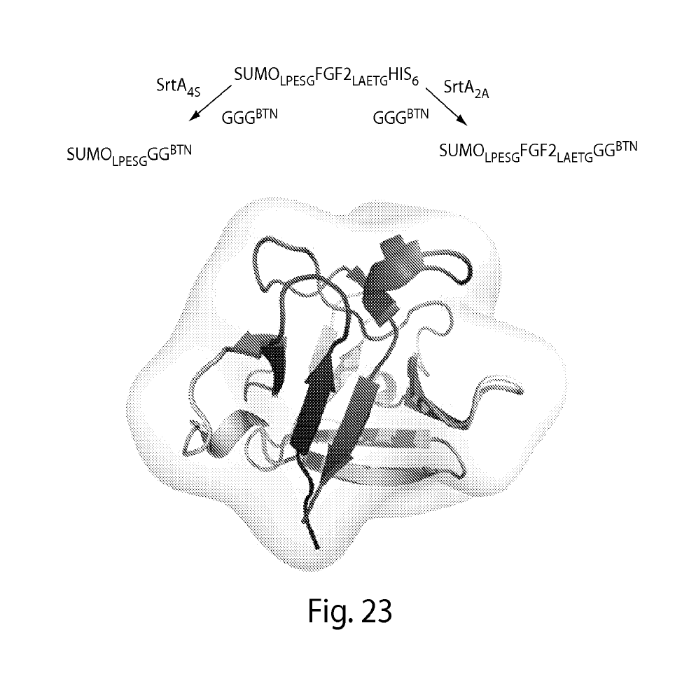

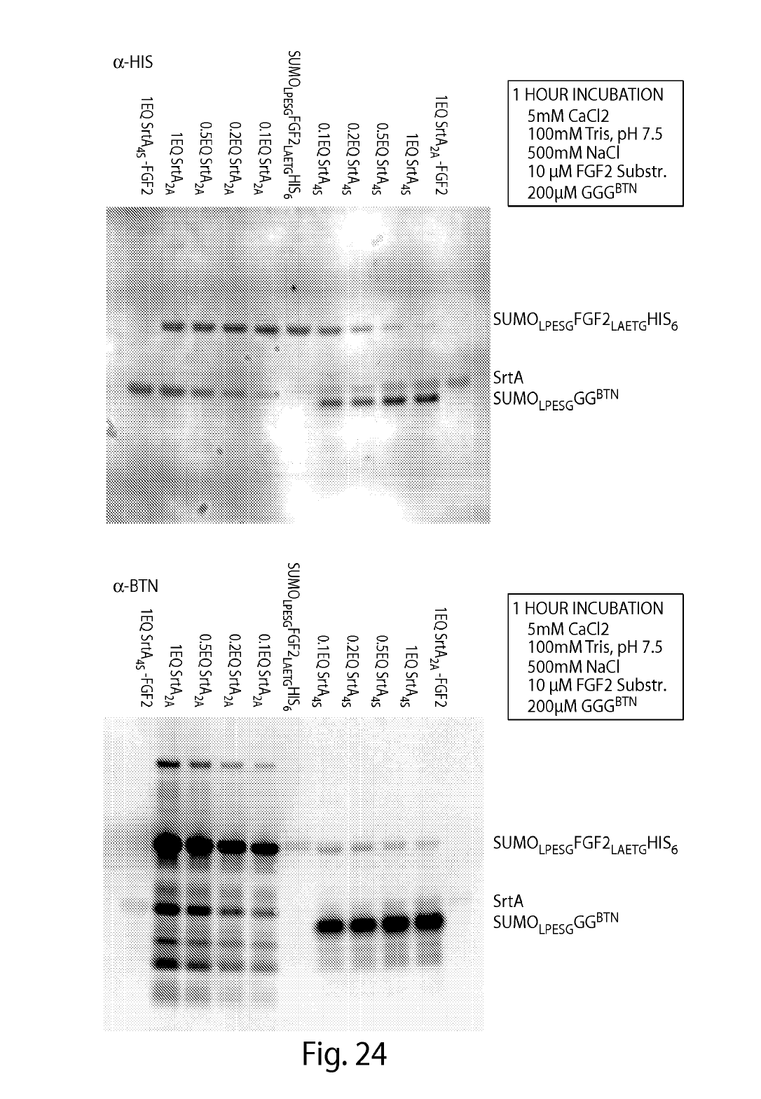

FGF2 is a relatively well-characterized protein that is involved in wound healing, among other functions. Chemically modified forms of FGF2 are currently in clinical trials. FGF2 is a relatively small, well-folded protein, with N- and C-termini being in close proximity in the folded protein (FIG. 23). The folded structure makes orthogonal site labeling of FGF2 challenging, since protein circularization competes very effectively with labeling when using conventional technologies. A sortase-mediated orthogonal modification scheme was developed, in which FGF2 was conjugated to a sumoyl moiety and a His6 tag via two different sortase recognition motifs, LPESG and LAETG, respectively (FIG. 23). After 1 h of incubation with various concentrations of two different sortases (2A and 4S), FGF2 samples were analyzed for hydrolyzation products (FIG. 24). Efficient orthogonal modification was observed.

All publications, patents, patent applications, publication, and database entries (e.g., sequence database entries) mentioned herein, e.g., in the Background, Summary, Detailed Description, and/or Examples sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1. A composition comprising,
an engineered solid support, and
a first sortase recognition motif conjugated to the surface of the solid support.

2. The composition of claim 1, wherein the solid support comprises a polymer or copolymer.

3. (canceled)

4. The composition of claim 2, wherein the polymer or co-polymer is chosen from the group consisting of polyurethane polymers and copolymers thereof, polyoxymethylene polymers and copolymers thereof, polyamide polymers and copolymers thereof, polyacrylamide polymers and copolymers thereof, polyvinyl polymers and copolymers thereof, styrene-ethylene-butylene copolymers, styrene-isoprene copolymers, polyether polymers and copolymers thereof, polyolefin polymers and copolymers thereof, polypropylene polymers and copolymers thereof, polyethylene polymers and copolymers thereof, polytetrafluoroethylene (PTFE) polymers and copolymers thereof, polyoxypropylene polymers and copolymers thereof, polyoxyethylene polymers and copolymers thereof, polyanhydride polymers and copolymers thereof, polyvinylalcohol polymers and copolymers thereof, and polyethyleneamine polymers and copolymers thereof.

5. The composition of claim 1, wherein the first sortase recognition motif comprises a polyglycine sequence.

6-7. (canceled)

8. The composition of claim 1, wherein the first sortase recognition motif comprises an LPXT motif, wherein X represents any amino acid.

9. The composition of claim 8, wherein the first sortase recognition motif comprises an LPETG (SEQ ID NO: 4) motif.

10. The composition of claim 1, wherein the solid support can be conjugated to a functional molecule conjugated to a second sortase recognition motif via a sortase-mediated transpeptidation, wherein the second sortase recognition motif can be used by a sortase as a substrate of a transpeptidation reaction involving the first sortase recognition motif.

11. The composition of claim 1, wherein the solid support is conjugated to a functional molecule comprising a second sortase recognition motif.

12. The composition of claim 10, wherein the composition comprises a [solid support]-[polyglycine sequence]-[LPXT]-[functional molecule] structure, or a [solid support]-[LPXT]-[polyglycine sequence]-[functional molecule] structure.

13. The composition of claim 1, wherein the solid support is in contact with a body fluid or tissue of a subject.

14. (canceled)

15. The composition of claim 11, wherein the functional molecule has anti-coagulant activity.

16. The composition of claim 15, wherein the functional molecule is thrombomodulin.

17. The composition of claim 1, wherein the composition is an implantable medical device.

18-20. (canceled)

21. The composition of claim 1, wherein the composition is in contact with a cell or cell population.

22-23. (canceled)

24. The composition of claim 11, wherein the functional molecule is a cell adhesion molecule.

25. The composition of claim 11, wherein the functional molecule is laminin or fibronectin.

26. The composition of claim 1, wherein the composition is in contact with an analyte in a sample.

27-31. (canceled)

32. The composition of claim 11, wherein the functional molecule is a binding molecule that specifically binds the analyte.

33. The composition of claim 32, wherein the functional molecule comprises an antibody or an antibody fragment.

34. (canceled)

35. A method comprising
contacting the composition of claim 1 with a sortase under conditions for the sortase to catalyze a transpeptidation reaction.

36. A composition comprising,
an engineered solid support, and
a functional molecule conjugated to the surface of the solid support via sortase-mediated transpeptidation.

37-48. (canceled)

49. A method for functionalizing a solid support surface, the method comprising
contacting a first sortase recognition motif conjugated to a solid support with a functional molecule conjugated to a second sortase recognition motif in the presence of a sortase and under conditions suitable for the sortase to catalyze a transpeptidation reaction conjugating the functional molecule to the solid support.

50-72. (canceled)

73. A method comprising
contacting a first functional molecule conjugated to a solid support comprising a first and a second sortase recognition motif with a sortase in the presence of a peptide comprising the first sortase recognition motif under conditions suitable for the sortase to catalyze a transpeptidation reaction that releases the first functional molecule and the second sortase recognition motif from the solid support and that restores the first sortase recognition motif conjugated to the solid support.

74-119. (canceled)
